SEQUENCE LISTING (15 sequences)

SEQ ID NO: 1 - protein, 20 aa, Escherichia coli
MKLLKVAAIAAIVFSGSALA

SEQ ID NO: 2 - protein, 20 aa, Escherichia coli
MKNKLLFMMLTILGAPGIAA

SEQ ID NO: 3 - protein, 22 aa, Escherichia coli
GVVPQYGGGGNHGGGGNNSGPN

SEQ ID NO: 4 - protein, 22 aa, Escherichia coli
AAGYDLANSEYNFAVNELSKSS

SEQ ID NO: 5 - protein, 42 aa, Escherichia coli
MKLLKVAAIAAIVFSGSALAGVVPQYGGGGNHGGGGNNSGPN

SEQ ID NO: 6 - protein, 42 aa, Escherichia coli
MKNKLLFMMLTILGAPGIAAAAGYDLANSEYNFAVNELSKSS

SEQ ID NO: 7 - protein, 151 aa, Escherichia coli
MKLLKVAAIAAIVFSGSALAGVVPQYGGGGNHGGGGNNSGPNSELNIYQYGGGNSALALQTDARNSDLTITQHGGGNGADVGQGSDDSSIDLTQRGFGNSATLDQWNGKNSEMTVKQFGGGNGAAVDQTASNSSVNVTQVGFGNNATAHQY

SEQ ID NO: 8 - protein, 151 aa, Escherichia coli
MKNKLLFMMLTILGAPGIAAAAGYDLANSEYNFAVNELSKSSFNQAAIIGQAGTNNSAQLRQGGSKLLAVVAQEGSSNRAKIDQTGDYNLAYIDQAGSANDASISQGAYGNTAMIIQKGSGNKANITQYGTQKTAIVVQRQSQMAIRVTQR

SEQ ID NO: 9 - protein, 72 aa, Artificial Sequence
  Description of Artificial Sequence Synthetic polypeptide
QQQQQQQQQQQQQQQQQQQQQQQQQQQQQQQQQQQQQQQQQQQQQQQQQQQQQQQQQQQQQQQQQQQQQQQQ

SEQ ID NO: 10 - protein, 25 aa, Artificial Sequence
  Description of Artificial Sequence Synthetic peptide
QQQQQQQQQQQQQQQQQQQQQQQQQ

SEQ ID NO: 11 - protein, 6 aa, Artificial Sequence
  Description of Artificial Sequence Synthetic 6xHis tag
HHHHHH

SEQ ID NO: 12 - protein, 10 aa, Artificial Sequence
  Description of Artificial Sequence Synthetic peptide
EQKLISEEDL

SEQ ID NO: 13 - protein, 8 aa, Artificial Sequence
  Description of Artificial Sequence Synthetic peptide
WSHPQFEK

SEQ ID NO: 14 - protein, 8 aa, Artificial Sequence
  Description of Artificial Sequence Synthetic peptide
DYKDDDDK

SEQ ID NO: 15 - protein, 14 aa, Artificial Sequence
  Description of Artificial Sequence Synthetic peptide
GKPIPNPLLGLDST

(19) United States
(12) Patent Application Publication
(10) Pub. No.: US 2015/0291991 A1
(43) Pub. Date: Oct. 15, 2015

(54) METHODS AND COMPOSITIONS RELATING TO AMYLOIDOGENIC POLYPEPTIDES

(71) Applicant: PRESIDENT AND FELLOW OF HARVARD COLLEGE, Cambridge, MA (US)

(72) Inventors: Ann Hochschild, Brookline, MA (US); Viknesh Sivanathan, Cambridge, MA (US)

(73) Assignee: PRESIDENT AND FELLOWS OF HARVARD COLLEGE, Cambridge, MA (US)

(21) Appl. No.: 14/441,925

(22) PCT Filed: Nov. 14, 2013
(86) PCT No.: PCT/US2013/070019

§ 371 Date: May 11, 2015

Related U.S. Application Data
(60) Provisional application No. 61/727,369, filed on Nov. 16, 2012.

(51) Int. Cl. C12P 21/02; C07K 14/395; C07K 14/245

(52) U.S. Cl. CPC C12P 21/02; C07K 14/245; C07K 14/395; C07K 2319/02

(57) ABSTRACT

The technology described herein relates to the expression of polypeptides, e.g. heterologous polypeptides using a bipartite curli signal sequence.

### CROSS-REFERENCE TO RELATED APPLICATIONS

This application claims benefit under 35 U.S.C. §119(e) of U.S. Provisional Application No. 61/727,369 filed Nov. 16, 2012, the contents of which are incorporated herein by reference in its entirety.

### GOVERNMENT SUPPORT

This invention was made with federal funding under Grant No. DP1AI104284 awarded by the National Institutes of Health. The U.S. government has certain rights in the invention.

### SEQUENCE LISTING

The instant application contains a Sequence Listing which has been submitted electronically in ASCII format and is hereby incorporated by reference in its entirety. Said ASCII copy, created on Nov. 12, 2013, is named 002806-072911-PCT_SL.txt and is 7,126 bytes in size.

### TECHNICAL FIELD

The technology described herein relates to the production of polypeptides and amyloid aggregates.

### BACKGROUND

Diverse proteins are known to be capable of forming amyloid aggregates, self-seeding fibrillar assemblies that may be biologically functional or pathological. Well known examples include neurodegenerative disease-associated proteins that misfold as amyloid, fungal prion proteins that can transition to a self-propagating amyloid form and certain bacterial proteins that fold as amyloid at the cell surface and promote biofilm formation. To further explore the diversity of amyloidogenic proteins as well as for studying the pathogenic mechanisms and potential therapeutics for pathogenic polypeptides, generally applicable methods for identifying, producing, and inducing amyloid formation are critical.

Among the protein misfolding diseases of the mammalian brain, the transmissible spongiform encephalopathies (TSEs) are singular because they are caused by infectious protein aggregates known as prions. Inevitably fatal, the TSEs are transmissible among humans, and from animals to humans. Prion infectivity is linked to conversion of a specific cellular protein, PrP, to a highly rugged amyloid aggregated state. PrP undergoes this conversion in vitro only with great difficulty, in the presence of denaturants, multiple cycles of sonication and incubation and/or facilitating factors to amplify the aggregated form.

### SUMMARY

In one aspect, described herein is a prokaryotic cell comprising: a nucleic acid sequence encoding a recombinant polypeptide, the recombinant polypeptide comprising, from 5′ to 3′ a bipartite curli signal sequence and a heterologous polypeptide sequence; wherein the bipartite curli signal sequence comprises, from 5′ to 3′ a SecA-dependent secretion signal and a CsgG targeting sequence. In some embodiments, the SecA-dependent secretion signal comprises the polypeptide sequence of SEQ ID NO: 1 (CsgA) or SEQ ID NO: 2 (CsgB). In some embodiments, the CsgG targeting sequence comprises the polypeptide sequence of SEQ ID NO: 3 or SEQ ID NO: 4. In some embodiments, the cell can further comprise a nucleic acid encoding a CsgG polypeptide wherein the CsgG polypeptide is expressed at ectopic expression levels. In some embodiments, the cell has been engineered to not transcribe or translate a csgA or csgB gene. In some embodiments, the cell is an Escherichia coli cell. In some embodiments, the heterologous polypeptide sequence is selected from the group consisting of: PrP; Aβ; α-synuclein; Sup35; the NM domain of Sup35; Rnq1; Cyc8; New1; Mss11; Pub1; Htt; exon 1 of Htt; NMRΔ; NMR2E2, FliE, Het-s; Tau; Superoxide dismutase 1; Htt with polyQ expansion; Htt exon 1 with polyQ expansion; ataxins with polyQ expansion; serum amyloid A; transthyretin; fibrinogen; fibrinogen α-chain; amylin (IAPP); amyloid aggregate-forming domains or fragments thereof; and mutants or variants thereof. In some embodiments, an anchor sequence comprised by the heterologous polypeptide sequence has been replaced with the CsgB anchor sequence. In some embodiments, the heterologous polypeptide sequence is provided with a C-terminal anchor sequence derived from CsgB. In some embodiments, the nucleic acid sequence encoding a recombinant polypeptide further comprises a protease cleavage site sequence located between the bipartite curli signal sequence and the heterologous polypeptide sequence. In some embodiments, the nucleic acid sequence encoding a recombinant polypeptide further comprises, from 5′ to 3′ an amyloidogenic peptide sequence and a protease cleavage site sequence located between the bipartite curli signal sequence and the heterologous polypeptide sequence. In some embodiments, the amyloidogenic peptide sequence specifies Sup35NM.

In one aspect, described herein is a library of a plurality of nucleic acid sequences encoding heterologous polypeptide sequences, the library comprising: a plurality of clonal prokaryotic cell populations; wherein each clonal population is comprised of prokaryotic cells as described herein; and wherein the clonal populations collectively comprise a plurality of nucleic acid sequences encoding heterologous polypeptide sequences. In one aspect, described herein is a library of a plurality of nucleic acid sequences encoding heterologous polypeptide sequences, wherein each nucleic acid sequence can encode a unique heterologous polypeptide sequence. In one aspect, described herein is a library of a plurality of heterologous polypeptide sequences, the library comprising: a plurality of populations of heterologous polypeptides; wherein each population of heterologous polypeptides is obtained according to the methods described herein. In some embodiments, each population comprises a unique heterologous polypeptide sequence.

In one aspect, described herein is a method of producing amyloidogenic polypeptides, comprising culturing a cell as described herein under conditions suitable for the expression and export of the recombinant polypeptide. In some embodiments, an extracellular amyloid polypeptide aggregate comprises the amyloidogenic polypeptides. In some embodiments, the cell is cultured under conditions that a) permit the expression and export of the recombinant polypeptide and b) permit the formation of amyloid aggregates. In some embodiments, the conditions that a) permit the expression and export of the recombinant polypeptide and b) permit the formation of amyloid aggregates comprise culturing the cell on a solid medium. In some embodiments, the cell is cultured under conditions that a) permit the expression and export of the recombinant polypeptide and b) inhibit the formation of amyloid aggregates. In some embodiments, the conditions that a) permit the expression and export of the recombinant polypeptide and b) inhibit the formation of amyloid aggregates comprise culturing the cell in a liquid medium. In some embodiments, the cell is cultured in medium comprising an amyloid facilitating factor. In some embodiments, the amyloid facilitating factor is selected from the group consisting of: RNA; polyanions; the synthetic anionic phospholipid POPG; lipids; and amyloidogenic polypeptide seed material.

In one aspect, described herein is a method of determining if a candidate polypeptide sequence comprises an amyloidogenic polypeptide, the method comprising; culturing a cell as described herein under conditions that permit the expression and export of the recombinant polypeptide; determining the presence or absence of amyloid aggregates; wherein the heterologous polypeptide sequence comprises the candidate polypeptide sequence; wherein the presence of amyloid aggregates indicates the candidate polypeptide sequence comprises an amyloidogenic polypeptide. In some embodiments, the cell is further cultured under conditions that permit the formation of amyloid aggregates. In some embodiments, the conditions that permit the formation of amyloid aggregates comprise culturing the cell on solid medium. In some embodiments, the cell is contacted with an amyloid-binding dye. In some embodiments, the amyloid-binding dye is selected from the group consisting of: Congo Red; BSB; K114; thioflavin T; thioflavin S; BTA-1; methoxy-XO4; and derivatives thereof. In some embodiments, the method further comprises subjecting a sample of the culture to a filter retention assay.

In one aspect, described herein is a method of identifying an amyloidogenic modulating agent, the method comprising; culturing a cell as described herein under conditions that permit the expression and export of the recombinant polypeptide; contacting the cell with a candidate agent; determining if the formation of amyloid aggregates is modulated; wherein a statistically significant difference in amyloid aggregation as compared to a reference indicates that the candidate agent is an amyloidogenic modulating agent. In some embodiments, the amyloidogenic modulating agent can modulate amyloid aggregation. In some embodiments, an amyloidogenic modulating agent can increase amyloid aggregation. In some embodiments, an amyloidogenic modulating agent can decrease amyloid aggregation. In some embodiments, the cell is cultured under conditions that a) permit the expression and export of the recombinant polypeptide and b) inhibit the formation of amyloid aggregates. In some embodiments, the conditions that a) permit the expression and export of the recombinant polypeptide and b) inhibit the formation of amyloid aggregates comprise culturing the cell in a liquid medium. In some embodiments, the cell can be cultured under conditions that permit the formation of amyloid aggregates (e.g., when seeking to identify an inhibitor of amyloid aggregation). In some embodiments, the heterologous polypeptide comprises a variant of an amyloidogenic polypeptide that forms amyloid aggregates at a lower or higher rate than the wild-type amyloidogenic polypeptide.

In one aspect, described herein is a method of identifying the presence of pathological amyloidogenic or amyloid-related material in a sample, the method comprising: culturing a cell as described herein under conditions that permit the expression and export of the recombinant polypeptide; contacting the cell with a sample; determining if the formation of amyloid aggregates is increased; wherein a statistically significant increase in amyloid aggregation as compared to a reference indicates that the sample comprises pathological amyloidogenic or amyloid-related material. In some embodiments, the heterologous polypeptide comprises a prion polypeptide or an amyloid aggregate-forming domain or fragment thereof. In some embodiments, the prion polypeptide is PrP. In some embodiments, the heterologous polypeptide comprises an amyloidogenic polypeptide or amyloid aggregate-forming domain or fragment thereof selected from the group consisting of: Aβ and α-synuclein. In some embodiments, the cell is cultured under conditions that a) permit the expression and export of the recombinant polypeptide and b) inhibit the formation of amyloid aggregates. In some embodiments, the conditions that a) permit the expression and export of the recombinant polypeptide and b) inhibit the formation of amyloid aggregates comprises culturing the cell in a liquid medium. In some embodiments, the sample is a biological sample obtained from a subject or an environmental sample.

In one aspect, described herein is a method of purifying a polypeptide of interest, the method comprising; culturing a cell as described herein in culture medium under conditions that permit the expression and export of the recombinant polypeptide; subjecting the cells and culture medium to centrifugation such that a non-cellular supernatant results; wherein the heterologous polypeptide sequence comprises the polypeptide of interest that is to be purified; wherein the SecA-dependent secretion signal is cleaved from the recombinant polypeptide during the export of the recombinant polypeptide; and wherein the supernatant resulting from centrifugation comprises soluble isolated polypeptide of interest. In some embodiments, the soluble isolated polypeptide of interest has the CsgG targeting sequence at its N-terminus. In some embodiments, the method comprises culturing the cell under conditions that permit the expression and export of the recombinant polypeptide; and wherein either the non-cellular supernatant or the supernatant resulting from centrifugation is contacted with a protease that can cleave the protease cleavage site; whereby the CsgG targeting sequence is cleaved from the polypeptide of interest. In some embodiments, the method comprises culturing the cell in culture medium under conditions that permit the expression and export of the recombinant polypeptide; and wherein either the non-cellular supernatant or the supernatant resulting from centrifugation is contacted with a protease that can cleave the protease cleavage site; whereby the CsgG targeting sequence and the amyloidogenic peptide are cleaved from the polypeptide of interest. In some embodiments, after the culturing step, the aggregation of exported extracellular recombinant polypeptide is induced. In some embodiments, the aggregation of exported extracellular recombinant polypeptide is induced by a method selected from the group consisting of: sonication and contacting with amyloidogenic seed material. Inducing protein aggregation can permit the aggregated material to be concentrated. In some embodiments, the resulting aggregates can be concentrated by centrifugation and the resuspended aggregates contacted with a protease that can cleave the protease cleavage site, liberating the polypeptide of interest. In some embodiments, the aggregates can be removed by centrifugation. In some embodiments, the polypeptide of interest comprises a purification tag. In some embodiments, the method further comprises a final step of purifying the polypeptide of interest from the supernatant resulting from centrifugation by means of the purification tag.

In one aspect, described herein is an isolated nucleic acid comprising, from 5′ to 3′ a bipartite curli signal sequence and an associated cloning site wherein the bipartite curli signal sequence comprises, from 5′ to 3′ a SecA-dependent secretion signal and a CsgG targeting sequence. In some embodiments, the SecA-dependent secretion signal comprises the polypeptide sequence of SEQ ID NO: 1 (CsgA) or SEQ ID NO: 2 (CsgB). In some embodiments, the CsgG targeting sequence comprises the polypeptide sequence of SEQ ID NO: 3 or SEQ ID NO: 4. In some embodiments, the cloning site is selected from the group consisting of: a multiple cloning site; a restriction enzyme site; and a TA cloning site. In some embodiments, an expression vector comprises the nucleic acid. In some embodiments, the expression vector is selected from the group consisting of: a plasmid and a phage vector. In some embodiments, a sequence encoding a polypeptide is inserted in the cloning site and wherein the polypeptide is selected from the group consisting of: PrP; Aβ; α-synuclein; Sup35; the NM domain of Sup35; Rnq1; Cyc8; New1; Mss11; Pub1; Htt; exon 1 of Htt; NMRΔ; NMR2E2, FliE, Het-s; Tau; Superoxide dismutase 1; Htt with polyQ expansion; Htt exon 1 with polyQ expansion; ataxins with polyQ expansion; serum amyloid A; transthyretin; fibrinogen; fibrinogen α-chain; amylin (IAPP); amyloid aggregate-forming domains or fragments thereof; and mutants or variants thereof. In some embodiments, the nucleic acid sequence further comprises a protease cleavage site sequence located between the bipartite curli signal sequence and the cloning site. In some embodiments, the nucleic acid further comprises, from 5′ to 3′, an amyloidogenic peptide sequence and a protease cleavage site sequence located between the bipartite curli signal sequence and the cloning site. In some embodiments, the amyloidogenic peptide sequence specifies Sup35NM.

In one aspect, described herein is a kit comprising; an isolated nucleic acid as described herein. In one aspect, described herein is a kit comprising an isolated nucleic acid as described herein, e.g. a nucleic acid comprising sequences encoding the bipartite CsgA signal sequence and a heterologous polypeptide and/or a cloning site for inserting a polypeptide-encoding sequence. In some embodiments, the isolated nucleic acid can be present in an expression vector. In one aspect, described herein is a kit comprising; an isolated nucleic acid as described herein; and a prokaryotic cell. In some embodiments, the prokaryotic cell further comprises, a nucleic acid encoding a CsgG polypeptide wherein the CsgG polypeptide is expressed at ectopic expression levels. In some embodiments, the cell has been engineered to not transcribe a csgA or csgB gene and/or to not produce a CsgA or CsgB polypeptide. In some embodiments, the cell is an Escherichia coli cell. In some embodiments, the kit can further comprise a medium. In some embodiments, the kit can further comprise a medium that will indicate the presence of amyloid aggregates and/or fibrils, e.g. the medium can comprise Congo Red.

### BRIEF DESCRIPTION OF THE DRAWINGS

FIG. 1 depicts images demonstrating the secretion and amyloid formation of ssCsgA-NM. FIG. 1 depicts a photomicrograph demonstrating that cells exporting the ssCsgA-NM fusion protein generate an abundance of fibers of varying dimensions as visualized by transmission electron microscopy. Scale bar: 100 nm

FIGS. 2A-2B demonstrate that amyloid-like aggregates are formed by secreted CsgAₛₛ-NM. FIG. 2A depicts electron micrographs of immunolabeled CsgAₛₛ-NM (left) and CsgAₛₛ-M (right) scraped cell samples. Fibrils are detected only with the CsgAₛₛ-NM sample. FIG. 2B depicts an image of the results of a filter retention assay demonstrating that CsgAₛₛ-NM, but not CsgAₛₛ-M, scraped cell samples contain SDS-resistant aggregates, as detected by filter retention, which are solubilized upon boiling.

FIGS. 3A-3B depict schematics of experiments that demonstrate that amyloid-like aggregates of CsgAₛₛ-NM are seeding-competent and infectious. FIG. 3A depicts the experiment in which E. coli cell extracts containing SDS-soluble NM-GFP were seeded with scraped cell samples (*) (CsgAₛₛ-NM, CsgAₛₛ-M, or CsgA) or with yeast extracts (**) prepared from a [PSI⁺] or [psi⁻] strain. Seed-only control samples consisted of the CsgAₛₛ-NM scraped cell sample or the IPSO yeast extract diluted into E. coli cell extracts containing overproduced GFP only. Samples from seeded reactions were removed at the indicated time points, treated with 2% SDS, and the presence of SDS-stable aggregates assayed by filter retention. SDS-stable aggregates that were retained were probed with an anti-GFP antibody. FIG. 3B depicts an experiment in which [pin⁻][psi⁻] yeast spheroplasts were infected with NM-GFP aggregates isolated by centrifugation from the seeding reactions at the 30 min time point and either sonicated (post-sonication) or not (pre-sonication). The results of both experiments are shown in the table at the bottom of the Figures.

FIGS. 4A-4B depict images of electron microscopy and filter retention assays demonstrating that amyloid-like aggregates are formed by other secreted yeast prion proteins. FIG. 4A depicts electron micrographs of scraped cell samples containing various yeast prion proteins as CsgAₛₛ-fusions. Fibrillar aggregates are detected for all samples. FIG. 4B depicts the results of a filter retention assay demonstrating that all samples contain SDS-resistant aggregates, as detected by filter retention, which are solubilized upon boiling.

FIGS. 5A-5B depict images of microscopy and filter retention assays demonstrating the aggregation propensity of CsgAₛₛ-NM variants. Fibril density within scraped cell samples (CsgAₛₛ-NM, CsgAₛₛ-M, or CsgAₛₛ-NMRΔ) as detected by EM (FIG. 5A) and the amount of SDS-resistant material as detected by filter retention (FIG. 5B) parallel the known amyloidogenicity of these protein variants, as does the colony color phenotype of the cells when plated on agar containing CR.

FIGS. 6A-6B depict images of microscopy and filter retention assays demonstrating amyloid aggregates formed by secreted CsgAₛₛ-Htt[exon 1]. FIG. 6A depicts electron micrographs of CsgAₛₛ-Htt72Q (“72Q” disclosed as SEQ ID NO: 9) (left) and CsgAₛₛ-Htt25Q (“25Q” disclosed as SEQ ID NO: 10) (right) scraped cell samples. Fibrils are detected only with the CsgAₛₛ-Htt72Q (“72Q” disclosed as SEQ ID NO: 9) sample. FIG. 6B depicts filter retention assay results demonstrating that CsgAₛₛ-Htt72Q (“72Q” disclosed as SEQ ID NO: 9), but not CsgAₛₛ-Htt25Q (“25Q” disclosed as SEQ ID NO: 10), scraped cell samples contain SDS-resistant aggregates, as detected by filter retention, which are not solubilized by boiling.

FIG. 7 depicts an image of the Western blot analysis of amounts of secreted CsgAss--NM, CsgAss--NMRΔ and CsgAss--M. Overnight cultures of VS16 transformed with compatible plasmids directing the inducible synthesis of CsgG and CsgAss--NM, CsgAss--NMRΔ or CsgAss--M were diluted to OD600 0.1 in 10 ml LB supplemented with the appropriate antibiotics (Carbenicillin 100 μg/ml; Chloramphenicol 25 μg/ml) and IPTG (1 mM) and grown at 37° C. to OD600 0.2. L--Arabinose (0.2% final conc.) was added and the cultures grown for an additional 4 hr. Cells were separated from the culture medium by centrifugation (4000 rpm for 10 min) and the supernatant incubated with 50 μl Ni--NTA (Qiagen) with gentle rocking at 4° C. for 16 hr. The supernatant was then separated from the Ni--NTA by centrifugation (2000 rpm for 3 min), washed with fresh LB (5 ml), and centrifuged again (2000 rpm for 3 min) Ni--NTA--bound protein was eluted in 100 μl 1×SDS loading buffer supplemented with 10 mM EDTA and subsequently examined for relative amounts by SDS--PAGE and Western blot. Blot, probed with anti--His6 (“His6” disclosed as SEQ ID NO: 11) antibody, shows comparable levels of CsgAss--NM and CsgAss--NMRΔ, and 4 to 16--fold more CsgAss--M.

FIG. 8 depicts an electron micrograph of scraped cell sample containing secreted CsgAss--NM that shows a dense meshwork of fibrillar aggregates.

FIG. 9 depicts an electron micrograph and filter retention assay results. Electron micrograph depicting scraped cell sample containing secreted CsgA (left). SDS--resistant aggregates, detected by filter retention assay, can be solubilized by treatment with formic acid (right). To prepare samples for treatment with SDS or formic acid, CsgA--producing cells that had been spotted on agar were scraped off of the plates in PBS (phosphate--buffered saline) and normalized to OD600 1.0 in a volume of 300 μl. BugBuster® Protein Extraction Reagent (Novagen), rlysozyme (Novagen) and OmniCleave endonuclease (Epicentre) were added to the unlysed cell suspension to final concentrations of 0.5×, 300 units/ml and 10 units/ml, respectively, followed by incubation at room temperature with gentle rocking for 15 min. The sample was then split into two equal aliquots and centrifuged (10,000×g for 15 min at 4° C.). Pelleted material was either resuspended in 2% SDS or dissolved in 90% formic acid. Formic acid was subsequently removed using a vacuum--fitted centrifuge (speedvac) and the lyophilized sample resuspended in 2% SDS and boiled for 20 min. Both samples were then tested for the presence of aggregates by filter retention.

FIG. 10 depicts an electron micrograph of scraped cell sample containing secreted CsgAₛₛ-FliE.

### DETAILED DESCRIPTION

The study of amyloidogenic proteins, and pathological conditions related thereto, has been stymied by the difficulty of obtaining and/or generating the amyloid forms of these proteins. Natural conversion to prions is usually a rare event and de novo conversion of purified PrP is a laborious process, requiring, e.g. multiple cycles of sonication and incubation and/or the presence of facilitating factors.

The export system described herein provides an efficient method for evaluating amyloid-forming potential without a need for protein purification. This can permit the evaluation of polypeptides and/or potential amyloidogenic promoting or inhibiting factors for their ability to form and/or induce amyloid aggregate formation. Notably, the technology described herein permits amyloidogenic polypeptides to be produced and converted to the amyloid form without the need for first purifying the polypeptides.

Described herein is a cell-based method for generating amyloid aggregates. Described here is the inventors' discovery of compositions and methods that adapt the curli export machinery of E. coli to express heterologous amyloidogenic polypeptides and promote their conversion to the amyloid form without the use of physical or chemical manipulation and without a requirement for facilitating factors (Further discussion of the curli export system can be found, e.g. in Robinson et al. 2006; which is incorporated by reference herein in its entirety). By engineering a cell to express a recombinant protein which comprises an N-terminal sequence which is compatible with the curli export system at least part of the recombinant protein can be transported across both the inner and outer membranes via the curli system. Export via the curli system is favorable to the formation of amyloid aggegates by amyloidogenic proteins, permitting the generation of amyloid aggregates. This technology has applications relating to the production of amyloid aggregates and amyloidogenic proteins as well as drug discovery and diagnostic purposes. The applications of this technology for producing amyloid aggregates of amyloidogenic proteins include the identification of amyloidogenic proteins, drug discovery and the implementation of bioassays for diagnostic purposes.

In one aspect, the technology described herein relates to a prokaryotic cell comprising a nucleic acid sequence encoding a recombinant polypeptide, the recombinant polypeptide comprising, from 5′ to 3′ a bipartite curli signal sequence and a heterologous polypeptide sequence. As used herein, a “bipartite curli signal sequence” refers to a nucleic acid sequence comprising, from 5′ to 3′ a SecA-dependent secretion signal and a CsgG targeting sequence.

As used herein, a “SecA-dependent secretion signal”, refers to a polypeptide sequence which, when present at the N-terminus of a polypeptide, can cause the polypeptide to be exported from the cytoplasm of a prokaryotic cell across the inner membrane. In some embodiments, the SecA-dependent secretion signal can be the first 20 amino acids of the bipartite curli signal sequence of an endogenous polypeptide exported by the curli export system. In some embodiments, the SecA-dependent secretion signal can be a polypeptide having the sequence of the E. coli CsgA SecA-dependent secretion signal (e.g. SEQ ID NO:1) and homologs and/or variants, including conservative substitution variants, thereof. In some embodiments, the SecA-dependent secretion signal can be a polypeptide having the sequence of the E. coli CsgB SecA-dependent secretion signal (e.g. SEQ ID NO:2) and homologs and/or variants, including conservative substitution variants, thereof.

As used herein, a “CsgG targeting sequence” refers to a polypeptide sequence which, when present at the N-terminus of a polypeptide, but C-terminal of the SecA-dependent secretion signal, can cause the polypeptide to be targeted to CsgG and exported across the outer membrane of the cell via the curli export system. In some embodiments, the CsgG targeting sequence can be the last 22 amino acids of the bipartite curli signal sequence of an endogenous polypeptide exported by the curli export system. In some embodiments, the CsgG targeting sequence can be a polypeptide having the sequence of the E. coli CsgA CsgG targeting sequence (e.g. SEQ ID NO:3) and homologs and/or variants, including conservative substitution variants, thereof. In some embodiments, the SecA-dependent secretion signal can be a polypeptide having the sequence of the E. coli CsgB CsgG targeting sequence (e.g. SEQ ID NO:4) and homologs and/or variants, including conservative substitution variants, thereof.

In some embodiments, the SecA-dependent secretion signal and CsgG targeting sequence of a bipartite curli signal sequence can be a naturally occurring combination of SecA-dependent secretion signal and CsgG targeting sequence, e.g. SEQ ID NOs: 1 and 3, i.e. SEQ ID NO: 5 or SEQ ID NOs: 2 and 4, i.e. SEQ ID NO: 6. In some embodiments, the SecA-dependent secretion signal and CsgG targeting sequence of a bipartite curli signal sequence can be from different genes, different species, and/or one or both can be variants, including conservative substitution variants of naturally occurring sequences. By way of non-limiting example, a SecA-dependent secretion signal comprising the sequence of SEQ ID NO: 1 can be combined with a CsgG targeting sequence comprising the sequence of SEQ ID NO: 4 to form a bipartite curli signal sequence. Alternatively, in a further non-limiting example, a SecA-dependent secretion signal comprising the sequence of SEQ ID NO: 2 can be combined with a CsgG targeting sequence comprising the sequence of SEQ ID NO: 3 to form a bipartite curli signal sequence.

As used herein, a “recombinant polypeptide” refers to a polypeptide comprising a 5′ portion comprising a bipartite curli signal sequence and a 3′ portion comprising a heterologous polypeptide sequence, i.e. a polypeptide sequence not naturally found operatively linked to a bipartite curli signal sequence. In some embodiments, the heterologous polypeptide sequence is foreign to the prokaryotic cell, e.g. not found in the genome of that species. In some embodiments, the heterologous polypeptide sequence is not homologous to a prokaryotic polypeptide sequence which is normally operatively linked to a SecA-dependent secretion signal or a CsgG targeting sequence.

As the export of the recombinant polypeptides described herein is dependent upon the curli export system, it can be advantageous to increase the expression of one or more components of the curli export system, e.g. by introducing a nucleic acid comprising a nucleic acid sequence encoding one or more components of the curli export system operatively linked to a promoter, e.g. a constitutive or inducible promoter. In some embodiments, a prokaryotic cell as described herein can comprise a nucleic acid encoding a CsgG polypeptide wherein the CsgG polypeptide is expressed at ectopic expression levels.

In some embodiments, the cell can have been engineered to not transcribe or translate a csgA or csgB gene.

Prokaryotic cells suitable for use in the compositions and methods described herein include any prokaryotic cells which comprise a curli export system. Non-limiting examples of such prokaryotic cells include Enterobacteriaceae spp., Salmonella enterica, Klebsiella spp., Escherichia spp., Enterobacteriaceae that form biofilms, E. coli, E. coli K12, and E. coli MG1655. (for further discussion see, e.g. Collinson et al. J Bacteriol 1993 1:12-8 and Zogaj et al. Infect Immun 2003 71:4151-8; which are incorporated by reference herein in their entireties). In some embodiments, the cell can be an Escherichia coli cell. Preferably, the prokaryotic cells are of a species and/or strain which is amenable to culture and genetic manipulation. In some embodiments, the parental strain of the prokaryotic cell of the technology described herein can be a strain optimized for protein expression. Non-limiting examples of bacterial species and strains suitable for use in the present technologies include Escherichia coli, E. coli BL21, E. coli Tuner, E. coli Rosetta, E. coli JM101, MC4100, and a csgBAC deletion derivative of any of the foregoing (e.g. a csgBAC deletion of MC4100) and derivatives of any of the foregoing. Bacterial strains for protein expression are commercially available, e.g. EXPRESS™ Competent E. coli (Cat. No. C2523; New England Biosciences; Ipswich, Mass.).

The recombinant polypeptide comprises a C-terminal heterologous polypeptide sequence. As used herein “heterologous polypeptide sequence” refers to any polypeptide sequence which is not homolgous to (e.g. a variant or homolog) of either CsgA or CsgB. A heterologous polypeptide sequence can comprise a prokaryotic or eukaryotic polypeptide sequence. A heterologous polypeptide sequence can comprise a complete polypeptide sequence, e.g. a polypeptide as normally expressed by a cell or organism, or fragments, variants, and/or domains thereof.

In some embodiments, a heterologous poplypeptide sequence can comprise an amyloidogenic polypeptide, a polypeptide known to form amyloid aggregates, a prion-forming polypeptide, a yeast prion polypeptide, a mammalian prion polypeptide, a human prion polypeptide, a yeast polypeptide, a mammalian polypeptide, a human polypeptide, and fragments, domains, and/or variants and mutants of any of the foregoing. In some embodiments, the heterologous polypeptide sequence can be selected from the group consisting of: PrP; Aβ; α-synuclein; Sup35; the NM domain of Sup35; Rnq1; Cyc8; New1; Mss11; Pub1; Htt; exon 1 of Htt; NMRΔ; NMR2E2, FliE, Het-s; Tau; Superoxide dismutase 1; Htt with polyQ expansion; Htt exon 1 with polyQ expansion, ataxins with polyQ expansion; serum amyloid A; transthyretin; fibrinogen; fibrinogen α-chain; amylin (IAPP); amyloid aggregate-forming domains or fragments thereof; and mutants or variants thereof. Non-limiting examples of amyloidogenic polypeptides and fragments and variants thereof are described, e.g. in Alberti et al. Cell 2009 137:146-158 (particularly those listed in Table S2) and Chiti and Dobson. Annu Rev Biochem 2006 75:333-366; each of which is incorporated by reference herein in its entirety.

A heterologous polypeptide, prion polypeptide, and/or amyloidogenic polypeptide can be from any source, e.g. a eukaryotic organism, a yeast, or a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits, hamsters, and bank voles. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the source organism is a mammal, e.g., a primate, e.g., a human. The mammal can be a human, non-human primate, mouse, rat, dog, cat, horse, or cow, but is not limited to these examples. In some embodiments, the heterologous polypeptide can be synthetic.

As used herein, “amyloidogenic” refers to an agent (e.g a peptide or a non-peptide) that either forms or increases the formation of amyloid aggregates.

In some embodiments, an anchor sequence comprised by the heterologous polypeptide sequence can be replaced with the CsgB anchor sequence. In some embodiments, the recombinant polypeptide can further comprise a CsgB anchor sequence.

In some embodiments, the nucleic acid sequence encoding a recombinant polypeptide further comprises a protease cleavage site sequence located between the bipartite curli signal sequence and the heterologous polypeptide sequence.

In some embodiments, the nucleic acid sequence encoding a recombinant polypeptide further comprises, from 5′ to 3′ an amyloidogenic peptide sequence and a protease cleavage site sequence located between the bipartite curli signal sequence and the heterologous polypeptide sequence. In some embodiments, the amyloidogenic peptide sequence specifies Sup35NM. In some embodiments, a linker polypeptide sequence can be located between the amyloidogenic peptide and the protease cleavage site.

In one aspect, the technology described herein relates to libraries of amyloidogenic polypeptides and/or peptides which can be screened and/or tested for amyloidogenic activity. In some embodiments, described herein is a library of a plurality of nucleic acid sequences encoding heterologous polypeptide sequences, the library comprising: a plurality of clonal prokaryotic cell populations; wherein each clonal population is comprised of prokaryotic cells as described herein; and wherein the clonal populations collectively comprise a plurality of nucleic acid sequences encoding heterologous polypeptide sequences. In some embodiments, described herein is a library of a plurality of heterologous polypeptide sequences, the library comprising: a plurality of populations of heterologous polypeptides; wherein each population of heterologous polypeptides is obtained according to the methods described herein. In some embodiments, each population can comprise a unique heterologous polypeptide sequence.

Methods of creating bacterial libraries, and/or libraries of compounds isolated from bacterial cells are well known in the art. By way of non-limiting example, a bacterial cell library can be in the form of a plurality of multi-well plates, with each well of a plate comprising a clonal bacterial population. The clonal bacterial populations can be provided in media (e.g. solid media or liquid media) or in glycerol stocks. In some embodiments, a library can comprise multiple wells which comprise identical clonal populations, i.e. a clonal population can appear multiple times in a library. In some embodiments, a library can comprise a plurality of multi-well plates, with each well of a plate comprising one or more heterologous polypeptide sequences isolated from one or more clonal bacterial populations. Methods of isolating polypeptides from bacterial cells are well known in the art and examples are described elsewhere herein. In some embodiments, libraries can be created using automated and/or high-throughput methods, e.g. robotic colony-picking.

In some embodiments, a library can comprise pooled samples, e.g. multiple clonal bacterial populations, multiple isolated heterologous polypeptides, or multiple isolated populations of heterologous polypeptides can be pooled so that a smaller number of samples must be initially screened. The individual components of a “positive” pool can be subsequently screened separately. In some embodiments, a pool can comprise as many as 30 clonal populations, e.g. 2 or more clonal populations, 10 or more clonal populations, 20 or more clonal populations, or 30 or more clonal populations. In some embodiments, a pool can comprise as many as 24 clonal populations.

In some embodiments, a library can comprise 10 or more pools of, populations of, and/or individual heterologous polypeptide species (e.g. isolated or present within bacterial cells), e.g. 10 or more, 100 or more, 1,000 or more, 10,000 or more, or 100,000 or more pools of, populations of, and/or individual heterologous polypeptide species.

In some embodiments, a nucleic acid encoding a recombinant polypeptide can be present within the prokaryotic genome, e.g. the nucleic acids can be incorporated into the genome. In some embodiments, a nucleic acid encoding a recombinant polypeptide can be present within a vector.

The term “vector”, as used herein, refers to a nucleic acid construct designed for delivery to a host cell or transfer between different host cells. As used herein, a vector can be viral or non-viral. Many vectors useful for transferring exogenous genes into target cells are available, e.g. the vectors may be episomal, e.g., plasmids, virus derived vectors or may be integrated into the target cell genome, through homologous recombination or random integration. In some embodiments, a vector can be an expression vector. As used herein, the term “expression vector” refers to a vector that has the ability to incorporate and express heterologous nucleic acid fragments in a cell. An expression vector may comprise additional elements, for example, the expression vector may have two replication systems, thus allowing it to be maintained in two organisms. The nucleic acid incorporated into the vector can be operatively linked to an expression control sequence when the expression control sequence controls and regulates the transcription and translation of that polynucleotide sequence.

In some embodiments, a nucleic acid encoding a recombinant polypeptide can be present within a portion of a plasmid. Plasmid vectors can include, but are not limited to, pBR322, pBR325, pACYC177, pACYC184, pUC8, pUC9, pUC18, pUC19, pLG339, pR290, pKC37, pKC101, SV 40, pBluescript II SK+/− or KS+/−(see “Stratagene Cloning Systems” Catalog (1993) from Stratagene, La Jolla, Calif, which is hereby incorporated by reference), pQE, pIH821, pGEX, pET series and derivatives thereof (see Studier et. al., “Use of T7 RNA Polymerase to Direct Expression of Cloned Genes,” Gene Expression Technology, vol. 185 (1990), which is hereby incorporated by reference in its entirety). Integrating vectors appropriate for use in the technologies described herein are known in the art and described, e.g. in Haldimann and Wanner, J Bact. 2001; which is incorporated by reference herein in its entirety. In some embodiments, the nucleic acid encoding a recombinant polypeptide can be present on an F′ episome.

As used herein, the term “viral vector” refers to a nucleic acid vector construct that includes at least one element of viral origin and has the capacity to be packaged into a viral vector particle. The viral vector can contain a transgenic (e.g. heterologous) gene in place of non-essential viral genes. The vector and/or particle may be utilized for the purpose of transferring any nucleic acids into cells either in vitro or in vivo. Numerous viral vectors are known in the art and can be used as carriers of a nucleic acid into a cell, e.g. lambda vector system gt11, gt WES.tB, Charon 4.

In some embodiments, the recombinant polypeptide can be constitutively expressed. In some embodiments, nucleic acids encoding the recombinant polypeptide can be operatively linked to a constitutive promoter. In some embodiments, the recombinant polypeptide can be inducibly expressed. In some embodiments, nucleic acids encoding the recombinant polypeptide can be operatively linked to an inducible promoter.

As described herein, an “inducible promoter” is one that is characterized by initiating or enhancing transcriptional activity when in the presence of, influenced by, or contacted by an inducer or inducing agent than when not in the presence of, under the influence of, or in contact with the inducer or inducing agent. In some embodiments, an “inducible promoter” is one that is characterized by initiating or enhancing transcriptional activity when in the presence of, influenced by, or contacted by an inducer or inducing agent relative to when not in presence of, under the influence of, or in contact with the inducer or inducing agent. An “inducer” or “inducing agent” may be endogenous, or a normally exogenous compound or protein that is administered in such a way as to be active in inducing transcriptional activity from the inducible promoter. In some embodiments, the inducer or inducing agent, e.g., a chemical, a compound or a protein, can itself be the result of transcription or expression of a nucleic acid sequence (e.g., an inducer can be a transcriptional repressor protein), which itself may be under the control of an inducible promoter. Non-limiting examples of inducible promoters include but are not limited to, the lac operon promoter, a nitrogen-sensitive promoter, an IPTG-inducible promoter, an arabinose-inducible promoter, a salt-inducible promoter, a tetracycline-inducible promoter, steroid-responsive promoters, rapamycin responsive promoters and the like. Inducible promoters for use in prokaryotic systems are well known in the art, see, e.g. the beta-lactamase, and lactose promoter systems (Chang et al., Nature, 275: 615 (1978, which is incorporated herein by reference); Goeddel et al., Nature, 281: 544 (1979), which is incorporated herein by reference), the arabinose promoter system, including the araBAD promoter (Guzman et al., J. Bacteriol., 174: 7716-7728 (1992), which is incorporated herein by reference; Guzman et al., J. Bacteriol., 177: 4121-4130 (1995), which is incorporated herein by reference; Siegele and Hu, Proc. Natl. Acad. Sci. USA, 94: 8168-8172 (1997), which is incorporated herein by reference), the rhamnose promoter (Haldimann et al., J. Bacteriol., 180: 1277-1286 (1998), which is incorporated herein by reference), the alkaline phosphatase promoter, a tryptophan (trp) promoter system (Goeddel, Nucleic Acids Res., 8: 4057 (1980), which is incorporated herein by reference), the P_{LtetO-1} and P_{lac/are-1} promoters (Lutz and Bujard, Nucleic Acids Res., 25: 1203-1210 (1997), which is incorporated herein by reference), and hybrid promoters such as the tac promoter (deBoer et al., Proc. Natl. Acad. Sci. USA, 80: 21-25 (1983), which is incorporated herein by reference herein in its entirety).

An inducible promoter useful in the methods and systems as disclosed herein can be induced by one or more physiological conditions, such as changes in pH, temperature, radiation, osmotic pressure, saline gradients, cell surface binding, and the concentration of one or more extrinsic or intrinsic inducing agents. The extrinsic inducer or inducing agent may comprise amino acids and amino acid analogs, saccharides and polysaccharides, nucleic acids, protein transcriptional activators and repressors, cytokines, toxins, petroleum-based compounds, metal containing compounds, salts, ions, enzyme substrate analogs, hormones, and combinations thereof. In specific embodiments, the inducible promoter is activated or repressed in response to a change of an environmental condition, such as the change in concentration of a chemical, metal, temperature, radiation, nutrient or change in pH. Thus, an inducible promoter useful in the methods and systems as disclosed herein can be a phage inducible promoter, nutrient inducible promoter, temperature inducible promoter, radiation inducible promoter, metal inducible promoter, hormone inducible promoter, steroid inducible promoter, and/or hybrids and combinations thereof. Appropriate environmental inducers can include, but are not limited to, exposure to heat (i.e., thermal pulses or constant heat exposure), various steroidal compounds, divalent cations (including Cu2+ and Zn2+), galactose, tetracycline, IPTG (isopropyl-(3-D thiogalactoside), as well as other naturally occurring and synthetic inducing agents and gratuitous inducers.

Inducible promoters useful in the methods and systems as disclosed herein also include those that are repressed by “transcriptional repressors” that are subject to inactivation by the action of environmental, external agents, or the product of another gene. Such inducible promoters may also be termed “repressible promoters” where it is required to distinguish between other types of promoters in a given module or component of the biological switch converters described herein. Preferred repressors for use in the present invention are sensitive to inactivation by physiologically benign agent. Thus, where a Lac repressor protein is used to control the expression of a promoter sequence that has been engineered to contain a lacO operator sequence, treatment of the host cell with IPTG will cause the dissociation of the Lac repressor from the engineered promoter containing a lacO operator sequence and allow transcription to occur. Similarly, where a tet repressor is used to control the expression of a promoter sequence that has been engineered to contain a tetO operator sequence, treatment of the host cell with tetracycline will cause the dissociation of the tet repressor from the engineered promoter and allow transcription of the sequence downstream of the engineered promoter to occur. As another non-limiting example, where a temperature sensitive variant of the bacteriophage lambda repressor is used to control the expression of a natural lambda promoter or a promoter sequence that has been engineered to contain a lambda operator sequence, a shift in temperature will cause the dissociation of the lambda repressor from the promoter and allow transcription of the sequence downstream of the promoter to occur.

In one aspect, described herein is a method of producing amyloidogenic polypeptides, comprising culturing the cell as described herein under conditions suitable for the expression and export of the recombinant polypeptide. Such conditions can include, but are not limited to, conditions under which the prokaryotic cell is capable of logarithmic growth and/or polypeptide synthesis. Conditions may vary depending upon the species and strain of prokaryotic cell selected. Conditions for the culture of prokaryotic cells are well known in the art. If the recombinant polypeptide is operatively linked to an inducible promoter, such conditions can include the presence of the suitable inducing molecule(s). In some embodiments, an extracellular amyloid polypeptide aggregate can comprise the amyloidogenic polypeptides.

In some embodiments, the cell can be cultured under conditions that a) permit the expression and export of the recombinant polypeptide and b) permit the formation of amyloid aggregates. Non-limiting examples of such conditions include culture on solid medium and/or in the presence of an amyloid facilitating factor. As used herein, “amyloid facilitating factor” refers to any factor and/or agent that increases the rate at which amyloid aggregation formation begins. Non-limiting examples include RNA; polyanions; the synthetic anionic phospholipid POPG; lipids; amyloidogenic polypeptide seed material; and facilitating factors known in the art, e.g. those discussed in Wang et al. Science 2010 327:1132; which is incorporated by reference herein in its entirety.

In some embodiments, the cell can be cultured under conditions that a) permit the expression and export of the recombinant polypeptide and b) inhibit the formation of amyloid aggregates. Non-limiting examples of such conditions include culturing the cell in liquid medium.

In one aspect, provided herein is a method of determining if a candidate polypeptide sequence comprises an amyloidogenic polypeptide, the method comprising; culturing a cell as described herein under conditions that permit the expression and export of the recombinant polypeptide; determining the presence or absence of amyloid aggregates; wherein the heterologous polypeptide sequence comprises the candidate polypeptide sequence; wherein the presence of amyloid aggregates indicates the candidate polypeptide sequence comprises an amyloidogenic polypeptide. In some embodiments, the cell can further be cultured under conditions that permit the formation of amyloid aggregates. In some embodiments, the cell can be contacted with an amyloid-binding dye. Non-limiting examples of amyloid-binding dye include Congo Red; BSB; K114; thioflavin T; thioflavin S; BTA-1; methoxy-XO4; and derivatives thereof. In some embodiments, thioflavin T and its derivatives can be used in liquid medium, e.g. to detect the kinetics of the formation of amyloid aggregates. Amyloid-binding dyes are known in the art, e.g. Crystal et al. J Neurochemistry 2003 86:1359-1368; which is incorporated by reference herein in its entirety.

In some embodiments, the method can further comprise subjecting a sample of the culture to a filter retention assay. Methods for performing a filter retention assay are described in the Examples herein.

The presence of amyloid aggregates can also be detected using methods such as SDD-AGE, Western blotting, TEM, and/or bright-field microscopy (to detect Congo Red birefringence), as described in the Examples herein.

In one aspect, described herein is a method of identifying an amyloidogenic modulating agent (i.e. an agent that increases or decreases the formation of amyloid aggregates), the method comprising; culturing a cell as described herein under conditions that permit the expression and export of the recombinant polypeptide; contacting the cell with a candidate agent; determining if the formation of amyloid aggregates is modulated; wherein a statistically significant difference in amyloid aggregation as compared to a reference indicates that the candidate agent is an amyloidogenic modulating agent. In one aspect, described herein is a method of identifying an amyloidogenic modulating agent of amyloid aggregation (i.e. an agent that increases or decreases the formation of amyloid aggregates), the method comprising; culturing a cell as described herein under conditions that permit the expression and export of the recombinant polypeptide; contacting the cell with a candidate agent; determining if the formation of amyloid aggregates is modulated; wherein a statistically significant difference in amyloid aggregation as compared to a reference indicates that the candidate agent is an amyloidogenic modulating agent. In one aspect, described herein is a method of identifying an amyloid aggregation modulating agent (i.e. an agent that increases or decreases the formation of amyloid aggregates), the method comprising; culturing a cell as described herein under conditions that permit the expression and export of the recombinant polypeptide; contacting the cell with a candidate agent; determining if the formation of amyloid aggregates is modulated; wherein a statistically significant difference in amyloid aggregation as compared to a reference indicates that the candidate agent is an amyloid aggregation modulating agent (e.g. an agent that modulates the aggregation of amyloid).

In some embodiments, the cell is cultured under conditions that a) permit the expression and export of the recombinant polypeptide and b) inhibit the formation of amyloid aggregates. In some embodiments, conditions that a) permit the expression and export of the recombinant polypeptide and b) inhibit the formation of amyloid aggregates can include culturing the cell in liquid medium. In some embodiments, conditions that a) permit the expression and export of the recombinant polypeptide and b) inhibit the formation of amyloid aggregates can include culturing the cell in salt and/or divalent ion concentrations that inhibit formation of amyloid aggregates. Such conditions can vary depending upon the sequence of the heterologous polypeptide.

In some embodiments, the heterologous polypeptide comprises a variant of an amyloidogenic polypeptide that forms amyloid aggregates at a lower rate than the wild-type amyloidogenic polypeptide.

In one aspect, described herein is a method of identifying the presence of pathological amyloidogenic material in a sample, the method comprising: culturing a cell as described herein under conditions that permit the expression and export of the recombinant polypeptide; contacting the cell with a sample; determining if the formation of amyloid aggregates is increased; wherein a statistically significant increase in amyloid aggregation as compared to a reference indicates that the sample comprises pathological amyloidogenic material. As described herein, “pathological amyloidogenic material” is amyloidogenic material that increases amyloid aggregation which is associated with, and/or symptomatic of, and/or causes a pathological condition. In some embodiments, the heterologous polypeptide comprises a prion polypeptide or an amyloid aggregate-forming domain or fragment thereof. In some embodiments, the prion polypeptide can be PrP. In some embodiments, the heterologous polypeptide can comprise an amyloidogenic polypeptide or amyloid aggregate-forming domain or fragment thereof selected from the group consisting of: Aβ and α-synuclein. In some embodiments, the cell can be cultured under conditions that a) permit the expression and export of the recombinant polypeptide and b) inhibit the formation of amyloid aggregates. In some embodiments, the sample is a biological sample obtained from a subject or an environmental sample. In some embodiments, the sample is a clinical sample, e.g. from a subject suspected of having a pathological condition related to amyloid aggregates and/or prions. Examples of pathological amyloidogenic material are described in, e.g. Chiti and Dobson. Annu Rev Biochem 2006 75:333-366 (particularly those listed in Table 1); which is incorporated by reference herein in its entirety.

In one aspect, described herein is a method of purifying a polypeptide of interest, the method comprising; culturing a cell as described herein in culture medium under conditions that permit the expression and export of the recombinant polypeptide; subjecting the cells and culture medium to centrifugation such that a non-cellular supernatant results; wherein the heterologous polypeptide sequence comprises the polypeptide of interest that is to be purified; wherein the SecA-dependent secretion signal is cleaved from the recombinant polypeptide during the export of the recombinant polypeptide; and wherein the supernatant resulting from centrifugation comprises soluble isolated polypeptide of interest. In some embodiments, the CsgG targeting sequence is present at the N-terminus of the soluble isolated polypeptide of interest.

In some embodiments, the method can comprise culturing a cell comprising a recombinant polypeptide comprising a protease cleavage site as described herein in culture medium under conditions that permit the expression and export of the recombinant polypeptide; and wherein either the non-cellular supernatant or the supernatant resulting from centrifugation is contacted with a protease that can cleave the protease cleavage site; whereby the CsgG targeting sequence is cleaved from the polypeptide of interest. In some embodiments, the method can comprise culturing the cell comprising a recombinant polypeptide comprising, from 5′ to 3′ an amyloidogenic peptide sequence and a protease cleavage site sequence located between the bipartite curli signal sequence and the heterologous polypeptide sequence, in culture medium under conditions that permit the expression and export of the recombinant polypeptide; and wherein either the non-cellular supernatant or the supernatant resulting from centrifugation is contacted with a protease that can cleave the protease cleavage site; whereby the CsgG targeting sequence and the amyloidogenic peptide are cleaved from the polypeptide of interest. In some embodiments, after the culturing step, the aggregation of exported extracellular recombinant polypeptide is induced. In some embodiments, the aggregation of exported extracellular recombinant polypeptide is induced by a method selected from the group consisting of: sonication and contacting with amyloidogenic seed material. An exported extracellular recombinant polypeptide can, in some embodiments, comprise a portion of a recombinant polypeptide, e.g. the portion of a recombinant polypeptide not comprising a SecA-dependent secretion signal. In some embodiments, the method of purifying a polypeptide of interest, comprises culturing a cell comprising a recombinant polypeptide comprising the bipartite CsgA signal sequence, followed by an amyloidogenic peptide (e.g. Sup35 NM), followed by a linker sequence, followed by a protease cleavage site, followed by the polypeptide of interest under conditions that permit the expression and export of the recombinant polypeptide; subjecting the cells and culture medium to centrifugation such that a non-cellular supernatant results; wherein the heterologous polypeptide sequence comprises the polypeptide of interest that is to be purified; wherein the supernatant resulting from centrifugation comprises soluble isolated polypeptide of interest; inducing the aggregation of the amyloidogenic peptide (e.g. either by sonication or the addition of an amyloid-inducing seed particle, e.g. an amyloidogenic seed particle); pelleting the aggregated material by centrifugation; resuspending the pelleted material in appropriate cleavage buffer and adding protease to release the polypeptide of interest; pelleting the aggregated material by centrifugation; wherein the supernatant resulting from centrifugation comprises soluble isolated polypeptide of interest. In some embodiments, the cleavage can be performed before inducing amyloid aggregation.

In some embodiments, the polypeptide of interest can comprise a purification tag. In some embodiments, the method can further comprise a final step of purifying the polypeptide of interest from the supernatant resulting from centrifugation by means of the purification tag. The term “purification tag” as used herein refers to any peptide sequence suitable for purification of a polypeptide. The purification tag specifically binds to (or is bound by) another moiety with affinity for the purification tag. Such moieties which specifically bind to a purification tag can be attached to a matrix or a resin, e.g. agarose beads. Moieties which specifically bind to purification tags can include antibodies, nickel or cobalt ions or resins, biotin, amylose, maltose, and cyclodextrin. Exemplary purification tags can include histidine tags (such as a hexahistidine peptide (SEQ ID NO: 11)), which will bind to metal ions such as nickel or cobalt ions. Therefore, in certain embodiments the purification tag can comprise a peptide sequence which specifically binds metal ions. Other exemplary purification tags are the myc tag (EQKLISEEDL (SEQ ID NO: 12)), the Strep tag (WSHPQFEK (SEQ ID NO: 13)), the FLAG tag (DYKDDDDK (SEQ ID NO: 14)) and the V5 tag (GKPIPNPLLGLDST (SEQ ID NO: 15)), the HA tag, and/or the VSV-G tag. The term “purification tag” also includes “epitope tags”, i.e. peptide sequences which are specifically recognized by antibodies. Exemplary epitope tags can include the FLAG tag, which is specifically recognized by a monoclonal anti-FLAG antibody. The peptide sequence recognized by the anti-FLAG antibody consists of the sequence DYKDDDDK (SEQ ID NO: 14) or a substantially identical variant thereof. Therefore, in certain embodiments the purification tag can comprise a peptide sequence which is specifically recognized by an antibody. The term “purification tag” also includes substantially identical variants of purification tags. “Substantially identical variant” as used herein refers to derivatives or fragments of purification tags which are modified compared to the original purification tag (e.g. via amino acid substitutions, deletions or insertions), but which retain the property of the purification tag of specifically binding to a moiety which specifically recognizes the purification tag.

In one aspect, described herein is an isolated nucleic acid comprising, from 5′ to 3′ a bipartite curli signal sequence and an associated cloning site, wherein the bipartite curli signal sequence comprises, from 5′ to 3′ a SecA-dependent secretion signal and a CsgG targeting sequence. As used herein, “a cloning site” refers to a position in a nucleic acid sequence that can accept the insertion of nucleic acid sequence(s), such that a polypeptide encoded by the inserted nucleic acid can be expressed, e.g. a sequence inserted at the cloning site will be operatively linked to a promoter as described herein. Non-limiting examples of a cloning site include a multiple cloning site; a restriction enzyme site; and a TA cloning site. An “associated cloning site” is a site positioned such that any nucleic acid inserted into the cloning site can be transcribed and translated as part of the same polypeptide that comprises the bipartitite curli signal sequence.

In some embodiments, the SecA-dependent secretion signal can comprise the polypeptide sequence of SEQ ID NO: 1 (CsgA) or SEQ ID NO: 2 (CsgB). In some embodiments, the CsgG targeting sequence can comprise the polypeptide sequence of SEQ ID NO: 3 or SEQ ID NO: 4.

In some embodiments, an expression vector comprises the nucleic acid. Non-limiting examples of an expression vector include a plasmid and a phage vector.

In some embodiments, a sequence encoding a polypeptide can be inserted in the cloning site and wherein the polypeptide is selected from the group consisting of: PrP; Aβ; α-synuclein; Sup35; the NM domain of Sup35; Rnq1; Cyc8; New1; Mss11; Pub1; Htt; exon 1 of Htt; NMRΔ; NMR2E2, FliE, Het-s; Tau; Superoxide dismutase 1; Htt with polyQ expansion; Htt exon 1 with polyQ expansion; ataxins with polyQ expansion; serum amyloid A; transthyretin; fibrinogen; fibrinogen α-chain; amylin (IAPP); amyloid aggregate-forming domains or fragments thereof; and mutants or variants thereof. In some embodiments, a sequence encoding an amyloidogenic polypeptide or a prion polypeptide, or a variant, mutant, and/or fragment or domain thereof can be inserted.

In some embodiments, the nucleic acid sequence further comprises a protease cleavage site sequence located between the bipartite curli signal sequence and the cloning site. In some embodiments, the nucleic acid can further comprise, from 5′ to 3′, an amyloidogenic peptide sequence and a protease cleavage site sequence located between the bipartite curli signal sequence and the cloning site. In some embodiments, the amyloidogenic peptide sequence specifies Sup35NM. A non-limiting example of a protease cleavage site is the TEV cleavage site. Protease cleavage sites and corresponding proteases are well known in the art (see, e.g. Simpson, R. J. Proteins and Proteomics. 2008 Cold Spring Harbor Laboratory Press; which is incorporated by reference herein in its entirety).

The methods and systems described herein depend on the successful export of the recombinant polypeptide (and/or at least the heterologous polypeptide) from the cytoplasm to the cell surface under the direction of the appended N-terminal signal sequences. In some embodiments, e.g. when testing a new recombinant polypeptide, growth conditions, host strain, etc, the methods described herein comprise growing suitably engineered bacteria (e.g. E. coli strains) on agar medium supplemented with the amyloid-binding dye, Congo Red. Successful export and conversion of the recombinant polypeptide (and/or at least the heterologous polypeptide) to the amyloid aggregated state at the cell surface results in bacterial colonies that stain red.

A lack of Congo Red staining of bacterial colonies can result from either 1) unsuccessful export of the recombinant polypeptide (and/or at least the heterologous polypeptide) or 2) failure of the recombinant polypeptide (and/or at least the heterologous polypeptide) that is exported to bind Congo Red. In order to be able to distinguish between these two possibilities, i.e. to determine if the recombinant polypeptide is being exported or not being exported, a reporter for successful export of the recombinant polypeptide may be employed. One non-limiting example of such a reporter system would rely on the activity of a bacterial protease that is fused to the test protein at its C-terminus, where any observable extracellular protease activity would be indicative of successful export of the recombinant polypeptide. Extracellular protease activity can be observed, e.g. as a zone of clearing (translucence) surrounding bacterial colonies when these colonies are grown on agar supplemented with a protein source, e.g. milk. One example of such a protease is ScNP, a zinc endoprotease produced by Streptomyces caespitosus. ScNP may be particularly suitable because it comprises only 132 amino acids (Kurisu et al., J Biochem 121: 304-308; 1997). A second non-limiting example of such a reporter system would rely on the activity of a phosphatase that is fused to the test protein, where any observable extracellular phosphatase activity would be indicative of successful export of the recombinant polypeptide. Extracellular phosphatase activity can be observed as a blue halo surrounding bacterial colonies when these colonies are grown on agar supplemented with 5-bromo-4-chloro-3-indolyl phosphate (XP), which turns blue after the phosphate moiety is cleaved. Additional reporter systems can rely on the activities of other enzymes that can be fused to the recombinant polypeptide; for each such activity, the bacteria can be grown on an appropriate solid medium that permits detection of a zone of activity surrounding colonies of cells exporting the enzyme. Suitable combinations of enzymes and media are known to one of skill in the art. Suitable enzymes and/or substrates are also available commercially, e.g. the ENZCHEK kits from LifeTechnologies (Grand Island, N.Y.) or the non-specific protease detection substrates available from Sigma-Aldrich (St. Louis, Mo.). Commonly used substrates include, but are not limited to milk proteins, casein, elastin, hemoglobin, BSA, and gelatin. Commonly used detectable signals can include a change in medium consistency and/or transperancy, a change in medium color, and fluorescence.

In some embodiments of any of the aspects described herein, a nucleic acid sequence described herein, e.g. one encoding a recombinant polypeptide, comprises from 5′ to 3′, a bipartite curli signal sequence, a heterologous polypeptide sequence, and a reporter enzyme. In some embodiments, the reporter enzyme is an enzyme that produces a detectable signal when it interacts with a substance present in the extracellular environment, e.g. an enzyme that produces a detectable signal when it interacts with a component of the medium. In some embodiments, the enzyme is not active and/or does not produce the detectable signal in the cytoplasm. In some embodiments, the reporter enzyme is a protease. In some embodiments, the reporter enzyme is a phosphatase. In some embodiments, the detectable signal is a visible “halo” around a bacterial colony, e.g. a change in color and/or media consistency and/or transparency. In some embodiments of any of the aspects described herein, the methods described herein can further comprise culturing a cell comprising a nucleic acid sequence comprising from 5′ to 3′, a bipartite curli signal sequence, a heterologous polypeptide sequence, and a reporter enzyme under conditions suitable for the expression and export of the recombinant polypeptide and suitable for detection of the activity of the reporter enzyme. In some embodiments, conditions suitable for the detection of the activity of the reporter enzyme can include culturing the cells in or on a medium comprising a substrate of the reporter enzyme, e.g. a substrate that when acted upon by the reporter enzyme, is converted to a detectable signal.

In one aspect, described herein is a kit comprising an isolated nucleic acid as described herein; and a prokaryotic cell. In some embodiments, the prokaryotic cell can further comprise a nucleic acid encoding a CsgG polypeptide wherein the CsgG polypeptide is expressed at ectopic expression levels. In some embodiments, the cell can be engineered to not transcribe a csgA or csgB gene. In some embodiments, the cell can be an Escherichia coli cell. In some aspect, described herein is a kit comprising an isolated nucleic acid as described herein, e.g. a nucleic acid comprising sequences encoding the bipartite CsgA signal sequence and a heterologous polypeptide and/or a cloning site for inserting a polypeptide-encoding sequence. In some embodiments, the isolated nucleic acid can be present in an expression vector. In one aspect, described herein is a kit comprising; an isolated nucleic acid as described herein; and a prokaryotic cell. In some embodiments, the prokaryotic cell further comprises, a nucleic acid encoding a CsgG polypeptide wherein the CsgG polypeptide is expressed at ectopic expression levels. In some embodiments, the cell has been engineered to not transcribe a csgA or csgB gene and/or to not produce a CsgA or CsgB polypeptide. In some embodiments, the cell is an Escherichia coli cell. In some embodiments, the kit can further comprise a medium. In some embodiments, the kit can further comprise a medium that will indicate the presence of amyloid aggregates and/or fibrils, e.g. the medium can comprise Congo Red.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, the terms “reduced”, “reduction”, “decrease”, or “inhibit” can mean a decrease by at least 10% as compared to a reference level, for example a decrease by at least about 20%, or at least about 30%, or at least about 40%, or at least about 50%, or at least about 60%, or at least about 70%, or at least about 80%, or at least about 90% or more or any decrease of at least 10% as compared to a reference level. In some embodiments, the terms can represent a 100% decrease, i.e. a non-detectable level as compared to a reference level. In the context of a marker or symptom, a “decrease” is a statistically significant decrease in such level. The decrease can be, for example, at least 10%, at least 20%, at least 30%, at least 40% or more, and is preferably down to a level accepted as within the range of normal for an individual without such disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operatively linked to appropriate regulatory sequences. A gene may or may not include regions preceding and following the coding region, e.g. 5′ untranslated (5′UTR) or “leader” sequences and 3′ UTR or “trailer” sequences, as well as intervening sequences (introns) between individual coding segments (exons).

The term “operatively linked” includes having an appropriate transcription start signal (e.g., promoter) in front of the polynucleotide sequence to be expressed, and having an appropriate translation start signal (e.g. Shine Delgarno and ATG) in front of the polypeptide coding sequence and maintaining the correct reading frame to permit expression of the polynucleotide sequence under the control of the expression control sequence, and, optionally, production of the desired polypeptide encoded by the polynucleotide sequence. In some examples, transcription of a gene encoding a recombinant polypeptide as described herein is under the control of a promoter sequence (or other transcriptional regulatory sequence) which controls the expression of the nucleic acid in a cell-type in which expression is intended. It will also be understood that the gene encoding a recombinant polypeptide as described herein can be under the control of transcriptional regulatory sequences which are the same or which are different from those sequences which control transcription of the naturally-occurring form of a protein.

The term “isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated.”

As used herein, the term “exogenous” refers to a substance (e.g. a nucleic acid or polypeptide) present in a cell other than its native source. The term exogenous can refer to a nucleic acid or a protein (that has been introduced by a process involving the hand of man into a biological system such as a cell or organism in which it is not normally found or in which it is found in undetectable amounts. A substance can be considered exogenous if it is introduced into a cell or an ancestor of the cell that inherits the substance. In contrast, the term “endogenous” refers to a substance that is native to the biological system or cell.

The term “agent” refers generally to any entity which is normally not present or not present at the levels being administered to a cell. An agent can be selected from a group comprising: polynucleotides; polypeptides; small molecules; antibodies; or functional fragments thereof.

As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide, e.g. the ability to target a polypeptide sequence to CsgG for export across the outer membrane. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. the ability to target a polypeptide sequence to CsgG for export across the outer membrane.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class.

Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, polypeptide described herein can be a variant of a sequence described herein, e.g. a conservative substitution variant of a polypeptide comprising the amino acid sequence of SEQ ID NO: 1. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity, e.g. ability to target a polypeptide for export via the curli export system. A wide variety of PCR-based site-specific mutagenesis approaches are also known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence, e.g. SEQ ID NO:1. The degree of homology (percent identity) between a native and a mutant sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A prokaryotic cell comprising:
   a nucleic acid sequence encoding a recombinant polypeptide, the recombinant polypeptide comprising, from 5′ to 3′ a bipartite curli signal sequence and a heterologous polypeptide sequence;
   wherein the bipartite curli signal sequence comprises, from 5′ to 3′ a SecA-dependent secretion signal and a CsgG targeting sequence.
2. The cell of paragraph 1, wherein the SecA-dependent secretion signal comprises the polypeptide sequence of SEQ ID NO: 1 (CsgA) or SEQ ID NO: 2 (CsgB).
3. The cell of any of paragraphs 1-2, wherein the CsgG targeting sequence comprises the polypeptide sequence of SEQ ID NO: 3 or SEQ ID NO: 4.
4. The cell of any of paragraphs 1-3, further comprising,
   a nucleic acid encoding a CsgG polypeptide wherein the CsgG polypeptide is expressed at ectopic expression levels.
5. The cell of any of paragraphs 1-4, wherein the cell has been engineered to not transcribe or translate a csgA or csgB gene.
6. The cell of any of paragraphs 1-5, wherein the cell is an Escherichia coli cell.
7. The cell of any of paragraphs 1-6, wherein the heterologous polypeptide sequence is selected from the group consisting of:
   PrP; Aβ; α-synuclein; Sup35; the NM domain of Sup35; Rnq1; Cyc8; New1; Mss11; Pub1; Htt; exon 1 of Htt; NMRΔ; NMR2E2, FliE, Het-s; Tau; Superoxide dismutase 1; Htt with polyQ expansion; Htt exon 1 with polyQ expansion; ataxins with polyQ expansion; serum amyloid A; transthyretin; fibrinogen; fibrinogen α-chain; amylin (IAPP); amyloid aggregate-forming domains or fragments thereof; and mutants or variants thereof
8. The cell of any of paragraphs 1-7, wherein an anchor sequence comprised by the heterologous polypeptide sequence has been replaced with the CsgB anchor sequence or the CsgB anchor sequence has been appended to the C-terminus of the heterologous polypeptide sequence.
9. The cell of any of paragraphs 1-8, wherein the nucleic acid sequence encoding a recombinant polypeptide further comprises a protease cleavage site sequence located between the bipartite curli signal sequence and the heterologous polypeptide sequence.
10. The cell of any of paragraphs 1-8, wherein the nucleic acid sequence encoding a recombinant polypeptide further comprises, from 5′ to 3′ an amyloidogenic peptide sequence and a protease cleavage site sequence located between the bipartite curli signal sequence and the heterologous polypeptide sequence.
11. The cell of paragraph 10, wherein the amyloidogenic peptide sequence specifies Sup35NM.
12. The cell of any of paragraphs 1-11, wherein the recombinant polypeptide further comprises a sequence encoding a reporter enzyme 3′ of the heterologous polypeptide.
13. The cell of paragraph 12, wherein the reporter enzyme is a protease or phosphatase.
14. A library of a plurality of nucleic acid sequences encoding heterologous polypeptide sequences, the library comprising:
   a plurality of clonal prokaryotic cell populations;
   wherein each clonal population is comprised of prokaryotic cells of any of paragraphs 1-13; and
   wherein the clonal populations collectively comprise a plurality of nucleic acid sequences encoding heterologous polypeptide sequences.
15. A library of a plurality of heterologous polypeptide sequences, the library comprising:
   a plurality of populations of heterologous polypeptides;
   wherein each population of heterologous polypeptides is obtained according to the methods of any of paragraphs 17-25 and 48-55.
16. The library of any of paragraphs 14-15, wherein each population comprises a unique heterologous polypeptide sequence.
17. A method of producing amyloidogenic polypeptides, comprising culturing the cell of any of paragraphs 1-13 under conditions suitable for the expression and export of the recombinant polypeptide.
18. The method of paragraph 17, wherein an extracellular amyloid polypeptide aggregate comprises the amyloidogenic polypeptides.
19. The method of paragraph 18, wherein the cell is cultured under conditions that a) permit the expression and export of the recombinant polypeptide and b) permit the formation of amyloid aggregates.
20. The method of paragraph 19, wherein the conditions that a) permit the expression and export of the recombinant polypeptide and b) permit the formation of amyloid aggregates comprise culturing the cell on a solid medium.
21. The method of paragraph 17, wherein the cell is cultured under conditions that a) permit the expression and export of the recombinant polypeptide and b) inhibit the formation of amyloid aggregates.
22. The method of paragraph 21, wherein the conditions that a) permit the expression and export of the recombinant polypeptide and b) inhibit the formation of amyloid aggregates comprise culturing the cell in a liquid medium.
23. The method of any of paragraphs 17-22, wherein the cell is cultured in medium comprising an amyloid facilitating factor.
24. The method of paragraph 23, wherein the amyloid facilitating factor is selected from the group consisting of:
   RNA; polyanions; the synthetic anionic phospholipid POPG; lipids; and
   amyloidogenic polypeptide seed material.
25. The method of any of paragraphs 17-23, wherein the method further comprises culturing the cell in a medium comprising a substrate of the reporter enzyme, wherein a detectable signal is produced by the action of the reporter enzyme on the substrate.
26. A method of determining if a candidate polypeptide sequence comprises an amyloidogenic polypeptide, the method comprising;
   culturing the cell of any of paragraphs 1-13 under conditions that permit the expression and export of the recombinant polypeptide;
   determining the presence or absence of amyloid aggregates;
   wherein the heterologous polypeptide sequence comprises the candidate polypeptide sequence;
   wherein the presence of amyloid aggregates indicates the candidate polypeptide sequence comprises an amyloidogenic polypeptide.
27. The method of paragraph 26, wherein the cell is further cultured under conditions that permit the formation of amyloid aggregates.
28. The method of paragraph 27, wherein the conditions that permit the formation of amyloid aggregates comprise culturing the cell on solid medium.
29. The method of any of paragraphs 26-28, wherein the cell is contacted with an amyloid-binding dye.
30. The method of paragraph 29, wherein the amyloid-binding dye is selected from the group consisting of:
   Congo Red; BSB; K114; thioflavin T; thioflavin S; BTA-1; methoxy-XO4; and derivatives thereof
31. The method of any of paragraphs 26-30, wherein the method further comprises subjecting a sample of the culture to a filter retention assay.
32. The method of any of paragraphs 26-31, wherein the method further comprises culturing the cell in a medium comprising a substrate of the reporter enzyme, wherein a detectable signal is produced by the action of the reporter enzyme on the substrate.
33. A method of identifying an amyloidogenic modulating agent or agent that modulates amyloid aggregation, the method comprising;
   culturing a cell of any of paragraphs 1-13 under conditions that permit the expression and export of the recombinant polypeptide;
   contacting the cell with a candidate agent;
   determining if the formation of amyloid aggregates is modulated;
   wherein a statistically significant difference in amyloid aggregation as compared to a reference indicates that the candidate agent is an amyloidogenic modulating agent or agent that modulates amyloid aggregation.
34. The method of paragraph 33, wherein the cell is cultured under conditions that a) permit the expression and export of the recombinant polypeptide and b) inhibit the formation of amyloid aggregates.
35. The method of paragraph 34, wherein the conditions that a) permit the expression and export of the recombinant polypeptide and b) inhibit the formation of amyloid aggregates comprise culturing the cell in a liquid medium.
36. The method of paragraph 33, wherein the cell is cultured under conditions that a) permit the expression and export of the recombinant polypeptide and b) permit the formation of amyloid aggregates.
37. The method of paragraph 36, wherein the conditions that a) permit the expression and export of the recombinant polypeptide and b) permit the formation of amyloid aggregates comprise culturing the cell on a solid medium.
38. The method of any of paragraphs 33-37, wherein the heterologous polypeptide comprises a variant of an amyloidogenic polypeptide that forms amyloid aggregates at a lower or higher rate than the wild-type amyloidogenic polypeptide.
39. The method of any of paragraphs 33-38, wherein the method further comprises culturing the cell in a medium comprising a substrate of the reporter enzyme, wherein a detectable signal is produced by the action of the reporter enzyme on the substrate.
40. A method of identifying the presence of pathological amyloidogenic material in a sample, the method comprising:
   culturing a cell of any of paragraphs 1-13 under conditions that permit the expression and export of the recombinant polypeptide;
   contacting the cell with a sample;
   determining if the formation of amyloid aggregates is increased;
   wherein a statistically significant increase in amyloid aggregation as compared to a reference indicates that the sample comprises pathological amyloidogenic material.
41. The method of paragraph 40, wherein the heterologous polypeptide comprises a prion polypeptide or an amyloid aggregate-forming domain or fragment thereof
42. The method of paragraph 41, wherein the prion polypeptide is PrP.
43. The method of paragraph 40, wherein the heterologous polypeptide comprises an amyloidogenic polypeptide or amyloid aggregate-forming domain or fragment thereof selected from the group consisting of:
   Aβ and α-synuclein.
44. The method of any of paragraphs 40-43, wherein the cell is cultured under conditions that a) permit the expression and export of the recombinant polypeptide and b) inhibit the formation of amyloid aggregates.
45. The method of paragraph 44, wherein the conditions that a) permit the expression and export of the recombinant polypeptide and b) inhibit the formation of amyloid aggregates comprises culturing the cell in a liquid medium.
46. The method of any of paragraphs 40-45, wherein the sample is a biological sample obtained from a subject or an environmental sample.
47. The method of any of paragraphs 40-46, wherein the method further comprises culturing the cell in a medium comprising a substrate of the reporter enzyme, wherein a detectable signal is produced by the action of the reporter enzyme on the substrate.
48. A method of purifying a polypeptide of interest, the method comprising;
   culturing the cell of any of paragraphs 1-13 in culture medium under conditions that permit the expression and export of the recombinant polypeptide;
   subjecting the cells and culture medium to centrifugation such that a non-cellular supernatant results;
   wherein the heterologous polypeptide sequence comprises the polypeptide of interest that is to be purified;
   wherein the SecA-dependent secretion signal is cleaved from the recombinant polypeptide during the export of the recombinant polypeptide; and
   wherein the supernatant resulting from centrifugation comprises soluble isolated polypeptide of interest.
49. The method of paragraph 48, wherein the method comprises culturing the cell of paragraph 9 in culture medium under conditions that permit the expression and export of the recombinant polypeptide; and
   wherein either the non-cellular supernatant or the supernatant resulting from centrifugation is contacted with a protease that can cleave the protease cleavage site;
   whereby the CsgG targeting sequence is cleaved from the polypeptide of interest.
50. The method of any of paragraphs 48-49, wherein the method comprises culturing the cell of any of paragraphs 10-11 in culture medium under conditions that permit the expression and export of the recombinant polypeptide; and
   wherein either the non-cellular supernatant or the supernatant resulting from centrifugation is contacted with a protease that can cleave the protease cleavage site;
   whereby the CsgG targeting sequence and the amyloidogenic peptide are cleaved from the polypeptide of interest.
51. The method of any of paragraphs 48-50; wherein after the culturing step, the aggregation of exported extracellular recombinant polypeptide is induced.
52. The method of paragraph 51, wherein the aggregation of exported extracellular recombinant polypeptide is induced by a method selected from the group consisting of:
   sonication and contacting with amyloidogenic seed material.
53. The method of any of paragraphs 48-52, wherein the polypeptide of interest comprises a purification tag.
54. The method of paragraph 53, wherein the method further comprises a final step of purifying the polypeptide of interest from the supernatant resulting from centrifugation by means of the purification tag.
55. The method of any of paragraphs 48-54, wherein the method further comprises culturing the cell in a medium comprising a substrate of the reporter enzyme, wherein a detectable signal is produced by the action of the reporter enzyme on the substrate.
56. An isolated nucleic acid comprising, from 5′ to 3′ a bipartite curli signal sequence and an associated cloning site,
   wherein the bipartite curli signal sequence comprises, from 5′ to 3′ a SecA-dependent secretion signal and a CsgG targeting sequence.
57. The nucleic acid of paragraph 56, wherein the SecA-dependent secretion signal comprises the polypeptide sequence of SEQ ID NO: 1 (CsgA) or SEQ ID NO: 2 (CsgB).
58. The nucleic acid of any of paragraphs 56-57, wherein the CsgG targeting sequence comprises the polypeptide sequence of SEQ ID NO: 3 or SEQ ID NO: 4.
59. The nucleic acid of any of paragraphs 56-58, wherein the cloning site is selected form the group consisting of:
   a multiple cloning site; a restriction enzyme site; and a TA cloning site.
60. The nucleic acid of any of paragraphs 56-59, wherein an expression vector comprises the nucleic acid.
61. The nucleic acid of paragraph 60, wherein the expression vector is selected from the group consisting of:
   a plasmid and a phage vector.
62. The nucleic acid of any of paragraphs 56-61, wherein a sequence encoding a polypeptide is inserted in the cloning site and wherein the polypeptide is selected from the group consisting of:
   PrP; Aβ; α-synuclein; Sup35; the NM domain of Sup35; Rnq1; Cyc8; New1; Mss11; Pub1; Htt; exon 1 of Htt; NMRΔ; NMR2E2, FliE, Het-s; Tau; Superoxide dismutase 1; Htt with polyQ expansion; Htt exon 1 with polyQ expansion; ataxins with polyQ expansion; serum amyloid A; transthyretin; fibrinogen; fibrinogen α-chain; amylin (IAPP); amyloid aggregate-forming domains or fragments thereof; and mutants or variants thereof
63. The nucleic acid of any of paragraphs 56-62, wherein the nucleic acid sequence further comprises a protease cleavage site sequence located between the bipartite curli signal sequence and the cloning site.
64. The nucleic acid of any of paragraphs 56-63, wherein the nucleic acid further comprises, from 5′ to 3′, an amyloidogenic peptide sequence and a protease cleavage site sequence located between the bipartite curli signal sequence and the cloning site.
65. The nucleic acid of paragraph 64, wherein the amyloidogenic peptide sequence specifies Sup35NM.
66. The nucleic acid of any of paragraphs 56-65, wherein the nucleic acid further comprises, 3′ of the cloning site, a nucleic acid sequence encoding a reporter enzyme.
67. A kit comprising;
   an isolated nucleic acid of any of paragraphs 56-66.
68. A kit comprising;
   an isolated nucleic acid of any of paragraphs 56-66; and
   a prokaryotic cell.
69. The kit of paragraph 68, wherein the prokaryotic cell further comprises,
   a nucleic acid encoding a CsgG polypeptide wherein the CsgG polypeptide is
   expressed at ectopic expression levels.
70. The kit of any of paragraphs 67-69, wherein the cell has been engineered to not transcribe a csgA or csgB gene.
71. The kit of any of paragraphs 67-70, wherein the cell is an Escherichia coli cell.
72. The kit of claim 71, wherein the isolated nucleic acid is present in an expression vector or plasmid.
73. The kit of any of claims 67-72, wherein the kit further comprises a growth medium, wherein the medium will display a detectable difference in the presence of an amyloid aggregate and/or fibril.
74. The kit of claim 73, wherein the detectable difference is a change in color.
75. The kit of claim 74, wherein the medium further comprises Congo Red.
76. The kit of any of paragraphs 67-75, wherein the kit further comprises a growth medium comprising a reporter enzyme substrate, wherein a detectable signal is produced by the action of the reporter enzyme on the substrate.

### EXAMPLES

### Example 1

Prions are infectious, self-propagating protein aggregates that have been implicated in a number of devastating neurodegenerative diseases that are transmissible among humans, and from animals to humans. Prion infectivity is linked to conversion of a specific cellular protein to an amyloid aggregated state. Despite intensive scientific attention, many fundamental questions about the processes that trigger amyloid aggregation remain to be answered. With no effective therapies available to alter prion disease course, new experimental avenues are crucial. The overall objective of the research described herein is to mobilize bacterial genetics as an experimental system to probe the behavior of amyloid proteins in a simplified cellular setting. In particular, described herein is a system to capitalize on two E. coli-based assays developed by the inventors (e.g. the use of the method described herein to assess whether or not a given polypeptide sequence is amyloidogenic (or to identify amyloidogenic polypeptides in a library) and to produce and/or aggregate such polypeptides and (ii) the use of the methods described herein to either identify modulators of amyloid aggregation and/or to detect the presence of infectious/amyloidogenic material in environmental or clinical samples).

As described herein, the ability of E. coli cells to assemble amyloid fibers at the cell surface has been exploited to develop a general assay for identifying amyloidogenic proteins. The E. coli surface-associated fibers are composed of two specific proteins, CsgA and CsgB, which are exported to the cell surface in an unfolded state by a dedicated export pathway. It is demonstrated herein that the export of heterologous amyloidogenic proteins (for example a yeast prion protein) through this pathway promotes their efficient conversion to the amyloid form. Based on these findings and the characteristics of this export pathway, this system can be adapted for the study of PrP. The de novo conversion of purified recombinant PrP to the infectious, aggregated form has been accomplished only recently and using a lengthy and cumbersome procedure. The E. coli-based system described herein provides a greatly simplified means to study the PrP conversion process and the effects of facilitating factors and other prospective modulators of PrP conversion and aggregation.

Prion diseases are transmissible not only among humans, but, alarmingly, also from animals to humans. Despite intensive scientific attention, no effective therapies for curing or even controlling prion diseases are available. Therefore, the development of new experimental avenues for probing the basic biology of prion diseases is crucial.

Despite 40 years of intensive scientific attention, no effective treatments have been developed for controlling mammalian prion diseases¹. Underlying these inevitably fatal neurodegenerative diseases is the specific cellular protein PrP, which has the potential to form self-propagating aggregates that are infectious. These aggregates are composed of highly structured β sheet-rich fibrils known as amyloids, and conversion to the fibrillar form (PrP^{res}) involves a specific change in the conformation of PrP²⁻⁴. Two important factors continue to hinder the development of therapeutics for prion disease. First, the mechanistic basis of prion-mediated cytotoxicity remains poorly understood. Although the majority of studies aimed at uncovering therapeutics for prion disease have targeted PrP^{res} (the protease resistant form of PrP), an increasing body of evidence points to pre-fibrillar aggregates of PrP as the most toxic species, suggesting that it may be critical to target the earliest steps in the conversion of soluble PrP to PrP^{res 1, 5-8}.

However, a major experimental challenge poses a considerable obstacle toward achieving this goal. Whereas many amyloidogenic proteins readily undergo conversion to the amyloid form in vitro, the de novo conversion of purified recombinant PrP to the infectious, aggregated form has been accomplished only with great difficulty^{5, 9-11}. Soluble PrP must typically be treated with denaturants to promote protein misfolding and subjected to multiple cycles of sonication and incubation (a procedure called Protein Misfolding Cyclic Amplification, or PMCA12) in the presence of facilitating factors to amplify the aggregated form. The requirement for these cumbersome manipulations complicates efforts to study the misfolding events or the intermediate oligomeric species generated during PrP aggregation.

To circumvent some of these difficulties, alternative amyloid-forming proteins are being explored as surrogate systems for the discovery of therapeutics for controlling prion diseases¹³. Described herein is an E. coli-based assay that facilitates the efficient fibrillization of a variety of amyloidogenic proteins at the cell surface.

E. coli cells produce adhesive cell surface-associated amyloid fibres known as curli that are implicated in biofilm formation. These fibres are composed of two specific proteins, CsgA and CsgB, which are exported to the cell surface by a dedicated export pathway¹⁴.

As described above, E. coli curli fibres are produced by export of the amyloidogenic proteins CsgA and CsgB to the cell surface. A dedicated export system is required for this process, and CsgA and CsgB are thought to be secreted through the export channel in an unfolded state^{14, 17} The characteristics of this export pathway led us to explore the possibility that it might facilitate the efficient conversion of heterologous amyloidogenic proteins to the amyloid form. Based on the results described below, described herein is a system which can permit the study of PrP. Without wishing to be bound by theory, the system may facilitate the conversion of PrP to PIT′ because (i) protein is exported in an unfolded state (recall that denaturants are required for the de novo conversion of soluble PrP to PrP in vitro) and (ii) refolding occurs in the presence of lipids at the cell surface. Note that lipids have been identified as facilitating factors in several in vitro conversion studies^{5, 9, 10}.

As an initial proof of principle, whether the well characterized yeast prion protein, Sup35, would form amyloid fibrils when exported to the E. coli cell surface was determined. The prion-forming domain of Sup35, NM, retains its ability to convert to the infectious, amyloid form when grafted onto heterologous proteins^{18, 19}. A fusion protein containing the export signal of CsgA (CsgA signal sequence) fused to the NM domain of Sup35 was created.

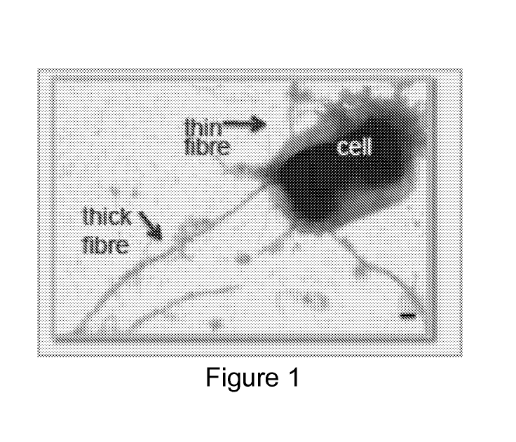

The resulting fusion protein (ssCsgA-NM) was then overproduced in a ΔcsgBA strain of E. coli, and the cells plated on agar medium supplemented with the amyloid-binding dye Congo Red, which stains the colonies red if the secreted protein is able to form amyloid aggregates at the cell surface¹⁴. Encouragingly, like cells producing wild-type curli fibers¹⁴, cells producing ssCsgA-NM stained deep red (data not shown). To confirm the presence of amyloid aggregates, colonies were scraped off of the plates and the material subjected to semi-denaturing detergent agarose gel electrophoresis (SDD-AGE)²⁰ and Western blotting to detect SDS-stable NM amyloid. The results indicate that export-mediated conversion of the NM fusion protein to the amyloid state is extremely efficient. Consistently, transmission EM revealed an abundance of fibrillar amyloid aggregates (FIG. 1). Interestingly, when overproduced in the E. coli cytoplasm, NM formed amyloid aggregates only in the presence of an inducing factor that is also required in yeast cells²¹. As expected, cells exporting an aggregation defective mutant of NM did not form red colonies and did not produce extracellular amyloid. 7 other amyloid-forming proteins were tested using this assay and all readily formed extracellular amyloid.

The ability of PrP from mouse, hamster and bank vole to convert to PrP^{res} can be assessed in the export system described herein. A naturally occurring single residue polymorphism for bank vole PrP, with one variant being conversion-deficient and the other being highly conversion-proficient, provides a particularly useful control. (In the long term, the respective animal models allow for assessment of the infectivity levels of any PrP generated). Synthesized prp genes (residues 23-230) that have been codon-optimized for expression in E. coli can be purchased and constructs carrying the csgA export signal generated. Using assays analogous to those described above for detecting NM amyloid aggregates, it can be determined if the exported PrP can access the PrP^{res} conformation. The presence of fibrillar aggregates of PrP can be determined visually by transmission EM. If amyloid aggregates of PrP are detected, these aggregates can be treated with Proteinase K, a diagnostic test used to detect the presence of the physiologically relevant aggregated form of PrP^{res 22, 23}. Whereas CsgA is normally secreted into the extracellular medium, CsgB is anchored in the outer membrane where it captures CsgA and triggers its conversion to the amyloid form²⁴. Because PrP is normally anchored at the cell surface via a C-terminal GPI membrane anchor, the effect of providing the CsgB C-terminal anchor sequence in place of the native GPI anchor sequence can be tested.

If the export system described herein does not support the spontaneous formation amyloid material, e.g. PrP^{res}, the effect of previously described facilitating factors can be tested. For example, a recent report describes the formation of infectious bacterially produced recombinant PrP^{res} in the presence of an anionic lipid and RNA by PMCA^{9, 10}. Thus, in an initial test cells producing cell surface associated and/or secreted PrP can be plated on RNA-containing medium²⁵. This E. coli-based system provides a greatly simplified means to study the effects of facilitating factors and other prospective modulators of PrP conversion and aggregation.

Finally, this system can be adapted as a sensitive and simple bioassay for detecting the presence of infectious material. Such an assay would most readily be carried out in a microtiter format in liquid medium, using cells producing secreted PrP. Without wishing to be bound by theory, under these conditions, the spontaneous conversion of exported PrP would likely be disfavored, at least in part due to the rapid diffusion of the exported molecules away from the producing cells. The presence of infectious material can be detected, e.g. by its ability to trigger the conversion of the secreted PrP into the aggregated form.

### REFERENCES

1. Trevitt C, Collinge J. 2006. A systematic survey of therapeutics in experimental models. Brain 129: 2241-2265
2. Aguzzi A, Polymenidou M. 2004. Mammalian prion biology: one century of evolving concepts. Cell 116(2): 313-27 Review
3. Aguzzi A, Sigurdson C, Heikenwaelder M. 2008. Molecular mechanisms of prion pathogenesis. Annu Rev Pathol 3: 11-40 Review
4. Pan K, Baldwin M, Nguyen J, Gasset M, Serban A, Groth D, Mehlhorn I, Huang Z, Fletterick R, Cohen F. 1993. Conversion of alpha-helices into beta-sheets features in the formation of the scrapie prion proteins. Proc Natl Acad Sci USA 90: 10962-10966
5. Caughey B, Baron G, Chesebro B, Jeffrey M. 2009. Getting a grip on prions: oligomers, amyloids, and pathological membrane interactions. Annu Rev Biochem 78: 177-204
6. Campioni S et. al. 2010. A causative link between the structure of aberrant protein oligomers and their toxicity. Nat Chem Biol 6, 140-147
7. Silveira J, and Raymond G, Hughson A, Race R, Sim V, Hayes S, Caughey B. 2005. The most infectious prion protein particles. Nature 437: 257-261
8. Chiti F, Dobson C. 2006. Protein misfolding, functional amyloid, and human disease. Annu Rev Biochem 75: 333-366
9. Wang F, Wang X, Yuan C, Ma J. 2010. Generating a prion with bacterially expressed recombinant prion protein. Sscience 327: 1132-1135
10. Wang F, Wang X, Ma J. 2011. Conversion of bacterially expressed recombinant prion protein. Methods 53: 208-213
11. Deleault N, Harris B, Rees J, Supattapone S. 2007. Formation of native prions from minimal components in vitro. Proc Natl Acad Sci USA 104: 9741-9746
12. Castilla J, Saà P, Hetz C, Soto C. 2005. In vitro generation of infectious scrapie prions. Cell 121: 195-206
13. Bach S et. al., Isolation of drugs active against mammalian prions using a yeast-based screening assay. 2003. Nat Biotechnol 21: 1075-1081
14. Chapman, M, Robinson L, Pinkner J, Roth R, Heuser J, Hammar M, Normark S, Hultgren S. 2002. Role of Escherichia coli curli operons indirecting amyloid fiber formation. Science 295: 851-855
15. Chernoff Y, Uptain S, Lindquist L. 2002. Analysis of prion factors in yeast. Methods Enzymol 351: 499-538
16. Bulic B, Pickhardt M, Khlistunova I, Biernat J, Mandelkow E, Mandelkow E, Waldmann H. 2007. Rhodanine-based tau aggregation inhibitors in cell models of tauopathy. Angew Chem Int Ed Engl 46: 9216-9219
17. Robinson L, Ashman E, Hultgren S, Chapman M. 2006. Secretion of curli fibre subunits is mediated by the outer membrane-localized CsgG protein. Mol Microbiol 59: 870-881
18. Uptain S, Lindquist S. 2002. Prions as protein-based genetic elements. Annu Rev Microbiol 56:703-741
19. Li L, Lindquist S. 2000. Creating a protein-based element of inheritance. Science 287:661-664
20. Bagriantsev S, Kushnirov V, Liebman S. 2006. Analysis of amyloid aggregates using agarose gel electrophoresis. Meth Enzymol 412: 33-48.
21. Garrity S, Sivanathan V, Dong J, Lindquist S, Hochschild A. 2010. Conversion of a yeast prion into an infectious form in bacteria. Proc Natl Acad Sci USA 107:10596-601.
22. Hope J, Morton L, Farquhar C, Multhaup G, Beyreuther K, Kimberlin R. 1986. The major polypeptide of scrapie-associated fibrils (SAF) has the same size, charge distribution and N-terminal protein sequence as predicted for the normal brain protein (PrP). EMBO J. 5:2591-97
23. Caughey B, Landsbury P. 2003. Protofibrils, pores, fibrils, and neurodegeneration: separating the responsible protein aggregates from the innocent bystanders. Annu Rev Neurosci 26: 267-298
24. Hammer N, Schmidt J, Chapman M. 2007. The curli nucleator protein, CsgB, contains an amyloidogenic domain that directs CsgA polymerization. Proc Natl Acad Sci USA 104: 12494-12499
25. Hole R, Singhal R, Melo J, D'Souza S. 2004. A rapid plate screening technique for extracellular ribonuclease producing strains. Barc Newsletter 249:91-97

### Example 2

Diverse proteins are known to be capable of forming amyloid aggregates, self-seeding fibrillar assemblies that may be biologically functional or pathological. Well known examples include neurodegenerative disease-associated proteins that misfold as amyloid, fungal prion proteins that can transition to a self-propagating amyloid form and certain bacterial proteins that fold as amyloid at the cell surface and promote biofilm formation. To further explore the diversity of amyloidogenic proteins, generally applicable methods for identifying them are critical. Described herein is a cell-based method for generating amyloid aggregates that relies on the natural ability of E. coli cells to elaborate amyloid fibrils at the cell surface. Several different yeast prion proteins and the human huntingtin protein are used to show that protein secretion via this specialized export pathway promotes acquisition of the amyloid fold specifically for proteins that have an inherent amyloid-forming propensity. Furthermore, the findings described herein establish the potential of this E. coli-based system to facilitate the implementation of high throughput screens for identifying amyloidogenic proteins and modulators of amyloid aggregation.

Diverse proteins from all domains of life are capable of forming amyloid aggregates made up of highly ordered β sheet-rich fibrils (Chiti and Dobson 2006). These fibrils share a characteristic cross-β spine, in which the β strands run perpendicular to the fibril axis (Toyama and Weissman 2011). Among protein aggregates, amyloid fibrils are unusually stable, typically exhibiting SDS resistance. A hallmark of amyloid aggregation is that it proceeds via a self-seeding mechanism, with a characteristic lag phase that can be eliminated by the addition of preformed fibrils (Chiti and Dobson 2006).

Among those proteins that are known to form amyloid aggregates under physiological conditions are the culprits in various devastating neurodegenerative diseases, including Alzheimer's, Parkinson's, Huntington's and the transmissible spongiform encephalopathies (TSEs) (Chiti and Dobson 2006). In addition to these disease-associated proteins that have a propensity to misfold as amyloid, mammalian proteins that assemble into amyloid aggregates to perform normal biological functions have been described. For example, various endocrine hormones are stored as amyloid aggregates in secretory granules (Maji et al. 2009).

Fungal prion proteins make up a particularly intriguing class of amyloidogenic proteins (Liebman and Chernoff 2012; Tuite and Serio 2010; Wickner et al. 2007). In general, fungal prion proteins have the potential to adopt alternative stable conformations, a so-called native fold and a self-propagating amyloid fold, which is the basis for prion formation. Often, but not in all cases, conversion to the prion form phenocopies a partial or full loss-of-function mutation. Although conversion to the prion form is typically a rare event, once formed, prions are stably transmitted from generation to generation and “infectious” when transferred to naïve strains. Thus, fungal prions act as non-Mendelian protein-based hereditary elements that can confer new phenotypic traits on the cells that harbor them.

In bacteria, all known amyloid-forming proteins aggregate extracellularly, in most cases forming surface-attached amyloid fibers (Blanco et al. 2011). In E. coli, these fibers, known as curli fibers, are composed of two proteins, CsgA and CsgB, that are directed to the outside of the cell by a dedicated export system (Blanco et al. 2011). It is demonstrated herein that the curli export apparatus can be appropriated for the production of extracellular amyloid fibers composed of heterologous amyloidogenic proteins derived from yeast and humans. These findings indicate that protein secretion through the curli export pathway facilitates acquisition of the amyloid fold specifically for proteins that have an inherent amyloid-forming propensity. The bacteria-based system described herein thus provides a simple and efficient means to distinguish amyloidogenic proteins from those that do not readily undergo conversion to an amyloid state.

Results

Experimental Plan.

The goal was to establish a generalizable cell-based system that would recapitulate aspects of widely employed in vitro assays for studying amyloid aggregation. Such in vitro assays involve purifying the protein of interest and subsequently monitoring its aggregation from a soluble and fully or partially unfolded state (see, for example, Wang et al. 2007). Without wishing to be bound by theory, it was hypothesized that a cell-based secretion system could similarly enable the separation of the protein of interest from the bulk of cellular protein in a fully or partially unfolded state that might facilitate acquisition of the amyloid fold. It was therefore sought to determine whether or not heterologous amyloid-forming proteins could be directed for export via the E. coli curli system and, if so, whether or not they would form extracellular amyloid fibrils.

Curli fibers are composed of two related amyloidogenic proteins: the major subunit CsgA and the minor subunit CsgB, which remains anchored in the outer membrane where it nucleates the polymerization of the fully secreted CsgA subunits (Chapman et al. 2002; Hammer et al. 2007; Blanco et al. 2011). Both CsgA and CsgB are translocated across the inner membrane into the periplasm by the general Sec-translocon system; subsequently, they are directed through a curli-specific pore-like structure in the outer membrane that is formed by the CsgG protein (Robinson et al. 2006). The specificity of this outer membrane secretion process depends on a 22 amino acid signal sequence at the N-terminus of the mature CsgA and CsgB proteins (Robinson et al. 2006). Under native conditions, curli biogenesis also depends on several accessory proteins (Blanco et al. 2011). Nevertheless, despite the complex requirements for curli biogenesis, previous work indicates that CsgG overproduction in the absence of all other curli factors enables the efficient secretion of CsgA, which does not assemble into amyloid fibrils, however, due to the absence of the nucleator CsgB (Chapman et al. 2002; Robinson et al. 2006). Thus, the strategy described herein for assessing the fate of heterologous amyloid-forming proteins directed to the curli export channel was to fuse the CsgA signal sequence to a set of target amyloidogenic proteins and overproduce these fusion proteins along with CsgG in a strain lacking CsgA and CsgB.

Yeast Prion Sup35 NM Forms Amyloid-Like Material when Exported from E. coli Using Curli System.

The well-characterized yeast prion protein Sup35 was first tested in this system. An essential translation release factor, Sup35 has a modular structure, with an N-terminal region (N) that contains the critical prion-forming determinants, a highly charged middle region (M) and a C-terminal domain (C) that carries out the translation release function (Glover et al. 1997; Liebman and Chernoff 2012). Together the N and M regions can function as a separable prion-forming module that is transferable to heterologous proteins (Li and Lindquist 2000). Accordingly, a plasmid vector was designed to direct the arabinose-inducible synthesis of Sup35 NM (hereafter NM) fused to the bipartite CsgA signal sequence (CsgAₛₛ), consisting of a SecA-dependent secretion signal (which is cleaved after passage through the Sec-translocon) and the CsgG targeting sequence (which is retained at the N-terminus of the mature protein). As a control, an otherwise identical plasmid directing the synthesis of the M domain (which lacks the essential prion-forming determinants and does not undergo conversion to an amyloid conformation; Glover et al. 1997) fused to the CsgAₛₛ was constructed.

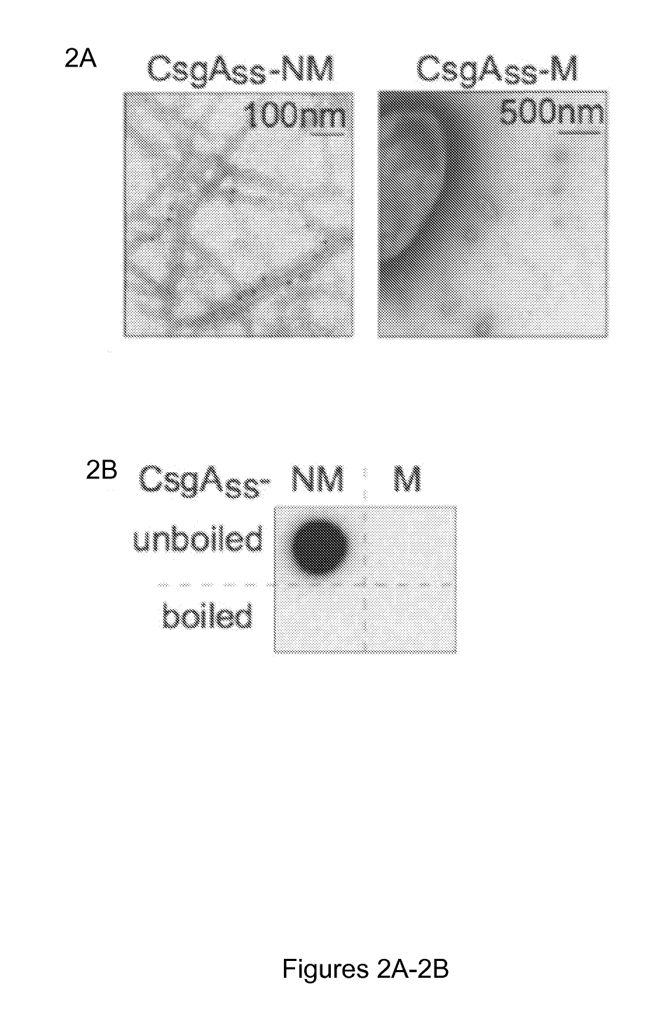

Each of these plasmids was introduced into a ΔcsgBAC strain of E. coli containing a second plasmid that directs the IPTG-inducible overproduction of CsgG. Cells containing either the NM plasmid or the M plasmid were plated onto inducing (i.e. arabinose+IPTG) medium containing Congo Red (CR), an amyloid-binding dye that can be used to detect the presence of curli fibers on E. coli cells (Hammar et al. 1995; Chapman et al. 2002). Like curli-positive cells of wild-type E. coli, cells producing CsgAₛₛ-NM formed colonies that stained bright red on this medium, whereas cells producing CsgAₛₛ-M formed pale colonies (data not shown). Furthermore, samples of the CsgAₛₛ-NM cells that had been plated on inducing medium revealed an abundance of fibrillar aggregates when examined by transmission EM, and the protein content of these fibrils was confirmed by immuno-gold labeling (FIG. 2A). In contrast, no such aggregates were observed in the case of the CsgAₛₛ-M cells. A control experiment indicated that the absence of CsgAₛₛ-M aggregates was not due to lower levels of secreted protein (FIG. 7). Bright field microscopy was also used to show directly that the CsgAₛₛ-NM fibrils bind CR and manifest ‘apple-green’ birefringence when examined between crossed polarizers (data not shown), a property that is diagnostic of amyloid material (Teng and Eisenberg 2009).

Another diagnostic characteristic of amyloid aggregates is their resistance to denaturation in the presence of SDS (Bagriantsev et al. 2006). To determine whether or not the aggregates produced by CsgAₛₛ-NM cells were SDS resistant, colonies (together with the fibrillar aggregates) were scraped off of inducing medium (without CR), the material resuspended in 2% SDS, and a filter retention assay (Alberti et al. 2009) used to test for the presence of SDS-stable NM aggregates (detectable with an anti-NM antibody). This analysis revealed an abundance of SDS-resistant aggregated material specifically with the CsgAₛₛ-NM cells that was solubilized when the samples were boiled (FIG. 2B).

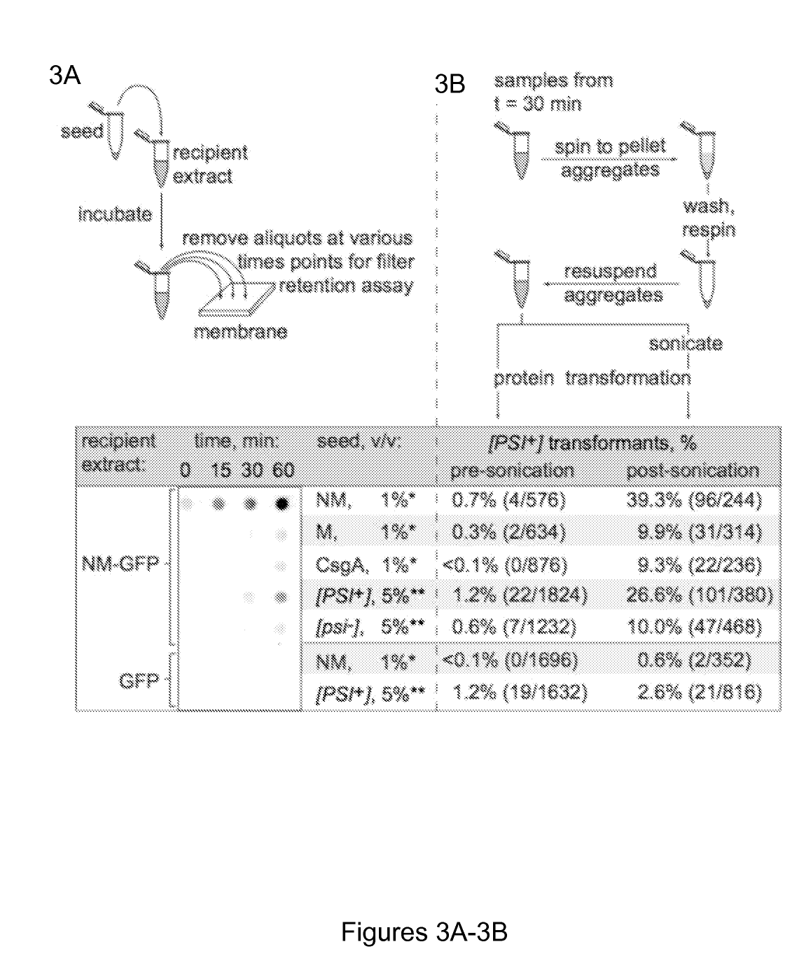

An additional key feature of amyloid is that aggregation proceeds via a self-seeding mechanism, with a characteristic lag phase that can be eliminated by the addition of preformed fibrils (Chiti and Dobson 2006). The ability of the scraped cell suspensions to seed the conversion of soluble NM protein to the amyloid aggregated (SDS-stable) form was tested. To carry out this test, the cell suspensions were diluted into extracts prepared from E. coli cells containing soluble NM-GFP fusion protein and the filter retention assay used to monitor the appearance of SDS-stable NM aggregates over time. These E. coli cell extracts support the slow, spontaneous conversion of NM-GFP to the amyloid form and this conversion reaction is accelerated in the presence of preassembled seed particles (Garrity et al. 2010). As expected based on these prior observations, the accumulation of a relatively small amount of SDS-stable NM aggregates was detected when the fibril-free CsgAₛₛ-M cell suspension was used as seed (FIG. 3A). However, the conversion reaction was significantly accelerated when the CsgAₛₛ-NM suspension was used as seed (FIG. 3A). As positive and negative controls, respectively, [PSI⁺] and [psi⁻] yeast extracts were used as seed (i.e. extracts prepared from yeast cells containing Sup35 in the prion and non-prion forms, respectively). A scraped cell suspension prepared from cells exporting native CsgA (see below) served as an additional negative control. Another pair of control reactions indicated that no SDS-stable NM aggregates accumulated when the seeding-competent samples were diluted into extract prepared from E. coli cells containing unfused GFP (empty extract).

Material Derived from Extracellular NM Aggregates Produced by E. coli can Induce Prion Formation when Introduced into Yeast Cells.

Having determined that cells exporting CsgAₛₛ-NM produce material with all the hallmarks of amyloid, it was sought to find out whether or not this amyloid-like material had accessed an infectious, prion conformation. To do this, a protocol for introducing exogenous prion aggregates into yeast cells and monitoring the conversion of Sup35 from the non-prion [psi⁻] form to the prion [PSI⁺] form (Tanaka and Weissman 2006) was used. Because [PSI⁺] cells are deficient in translation termination and manifest a heritable nonsense suppression phenotype, they can readily be distinguished from [psi⁻] cells on appropriate indicator medium. Importantly, the spontaneous conversion of Sup35 to the prion form in yeast cells is strictly dependent on the presence of a so-called [PSI⁺] inducibility (PIN) factor, which is itself a prion (Derkatch et al. 1997; Derkatch et al. 2001; Osherovich and Weissman 2001). Thus, the use of a [pin⁻] strain ensures that only seeded conversion events are detected. Accordingly, scraped cell suspensions of the E. coli strains described herein (supplemented with plasmid DNA encoding a yeast selectable marker) were tested for infectivity by using them to transform yeast spheroplasts prepared from a suitably marked [pin⁻] [psi⁻] yeast strain. [PSI⁺] transformants were obtained when the CsgAₛₛ-NM cells were used (at a frequency of 0.4%), but not when the CsgAₛₛ-M cells were used (at a frequency of <0.05%) (Table 1).

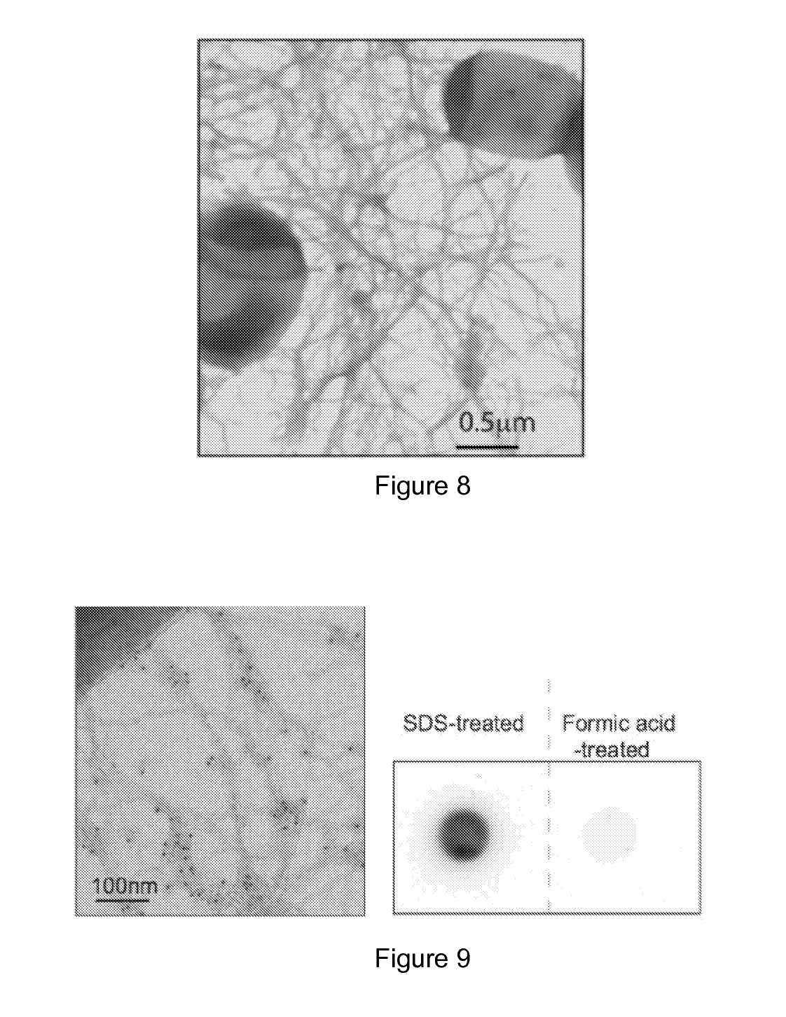

It was suspected that this relatively low [psi⁻] to [PSI⁺] conversion frequency was attributable to the fact that the fibrillar material, which appeared as a dense meshwork of long fibers (FIG. 8), likely was inefficiently taken up by the yeast cells and, once internalized, may have provided relatively few free ends to nucleate the polymerization of soluble Sup35. In fact, in vitro generated Sup35 aggregates are typically fragmented by sonication to increase their infectivity (King and Diaz-Avaos 2004; Tanaka and Weissman 2006). However, because sonication can also stimulate the assembly of soluble NM into amyloid aggregates and because the extracellular fibrils could not be completely separated from intact cells, it was not possible to simply test whether sonication enhanced the infectivity of the fibrillar material in the scraped cell suspensions. As an alternative strategy, the infectivity of the material generated in the seeding reactions of FIG. 3A was assessed. To do this, the high-molecular-weight aggregates were isolated from the seeding reactions (at the 30 minute time point) by centrifugation and tested for infectivity before and after sonication (FIG. 3B). That is, the isolated aggregates were used to transform [pin⁻] [psi⁻] yeast spheroplasts, as described above. In accord with previous observations using E. coli cell extracts containing soluble NM-GFP as substrate for the conversion reaction (Garrity et al. 2010), sonication dramatically increased the infectivity of the aggregates. Furthermore, the seeded reactions exhibited a marked increase in infectivity as compared with the mock seeded reactions. Thus, whereas the mock seeded reactions (containing the CsgAₛₛ-M suspension, the CsgA suspension, or the [psi⁻] yeast extract as seed) resulted in conversion frequencies of 9 to 10%, the reactions seeded with the CsgAₛₛ-NM suspension and the [PSI⁺] extract resulted in conversion frequencies of 39% and 27%, respectively. These results indicate that E. coli cells exporting CsgAₛₛ-NM produce amyloid-like material that is capable of seeding the conversion of soluble NM to an infectious, prion conformation.

Curli-Based Export System Provides a General Method for Detecting Amyloid-Forming Potential.

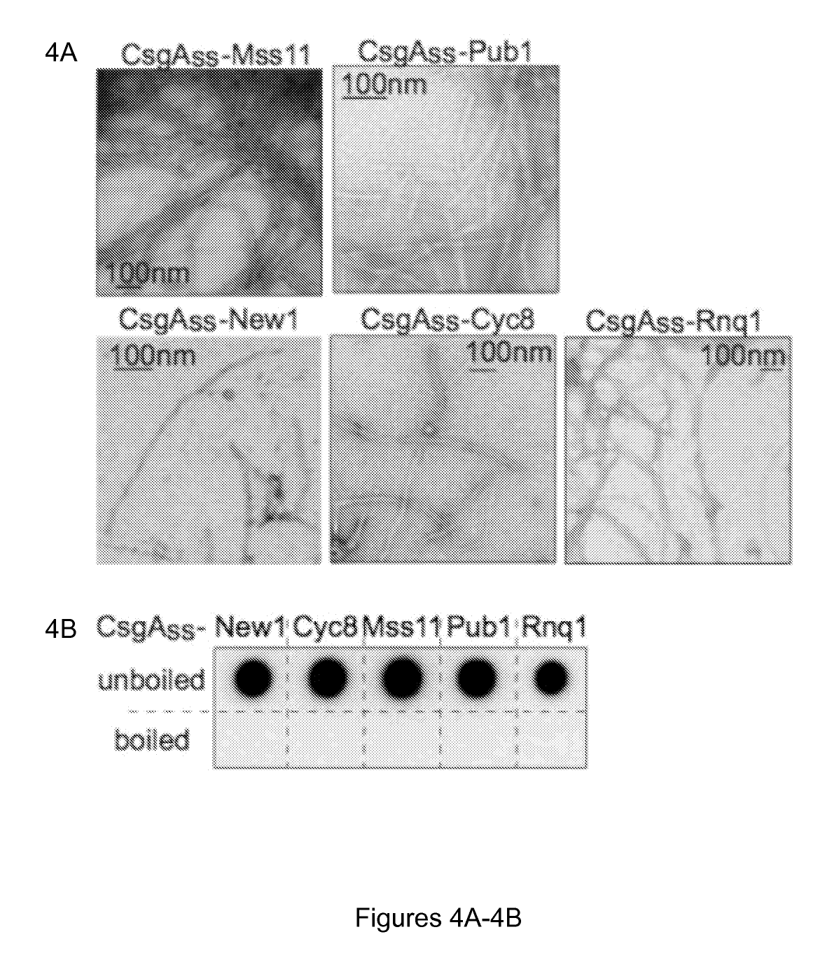

To evaluate the generality of these findings with CsgAₛₛ-NM, three other yeast prion proteins (Rnq1, Cyc8 and New1) and two candidate prion proteins (Mss11 and Pub1) were tested, all of which have been shown to form amyloid aggregates in vitro (Alberti et al. 2009). In each case, the previously defined prion-forming domain (Alberti et al. 2009) was fused to the CsgAₛₛ and a His₆-tag provided at the C-terminus of the fusion protein. As for CsgAₛₛ-NM, transmission EM of cell samples scraped from inducing medium revealed extracellular fibrillar aggregates in each case (FIG. 4A). Furthermore, the aggregates (detected with an antibody to the C-terminal His-tag) were SDS-resistant, as determined by the filter retention assay, but were solubilized when the material was boiled (FIG. 4B). CsgA itself (with a C-terminal His-tag (SEQ ID NO: 11)) was tested and fibril formation and the presence of SDS-resistant aggregates was observed (FIG. 9), indicating that under the experimental conditions described herein, the local concentration of exported CsgA is sufficiently high to allow polymerization in the absence of the CsgB nucleator (Hammer et al. 2007).

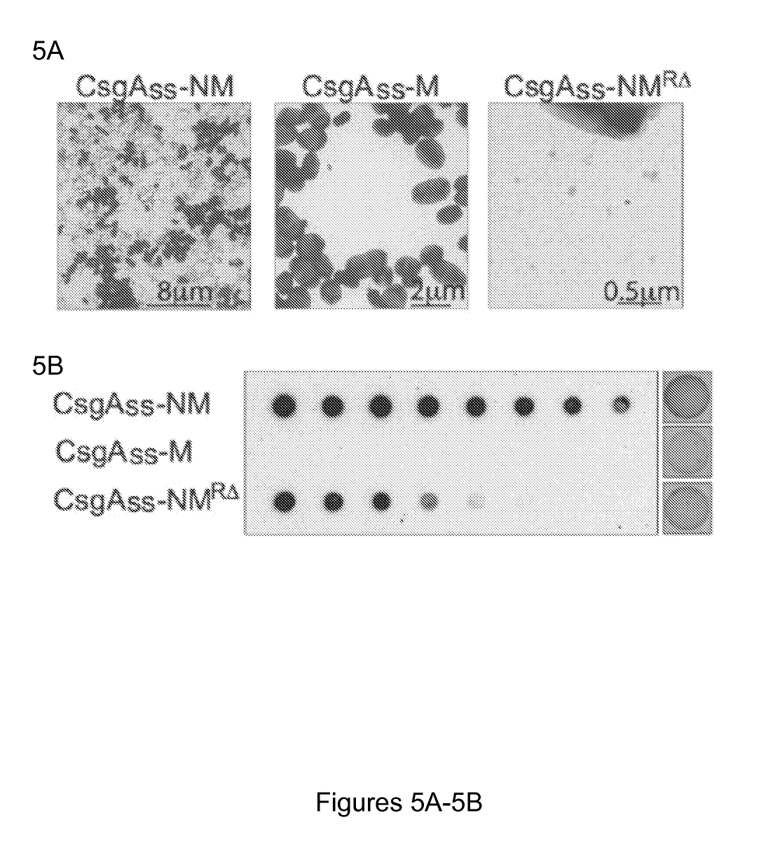

In principle, the curli-based export system described herein might provide a convenient means to screen for modulators of amyloid aggregation provided it was sufficiently sensitive to distinguish more or less conversion-prone variants of a single amyloid-forming protein. To further evaluate the sensitivity of the system, cells producing CsgAₛₛ-NM, CsgAₛₛ-NM^{RΔ} (an NM variant lacking 4 of 5 critical oligopeptide repeat sequences that has a greatly reduced ability to undergo spontaneous conversion to the prion form; Liu and Lindquist 1999), and CsgAₛₛ-M were compared. Cells exporting CsgAₛₛ-NM^{RΔ} produced extracellular fibrils, but they were both thinner and far less abundant than those produced by the CsgAₛₛ-NM cells (FIG. 5A). Consistently, comparison of the scraped cell samples revealed that the CsgAₛₛ-NM^{RΔ} sample contained much less SDS-stable NM material than the CsgAₛₛ-NM sample (FIG. 5B). These observations were paralleled by the colony colors as visualized on inducing medium containing CR, with the CsgAₛₛ-NM^{RΔ} cells staining a pale shade of red (FIG. 5B) despite secreting a similar amount of fusion protein as the CsgAₛₛ-NM cells (FIG. 7). Together, these findings indicate that this curli-based system could be adapted for the identification of modulators of amyloid aggregation. While colony color can effectively be used to report on the amount of amyloid formed by a particular protein and its variants, colony color cannot be used as a general surrogate for the presence of amyloid-like material because CR binding varies depending on the particular protein (Teng and Eisenberg 2009). In fact, when cells producing each of the 5 other yeast prion proteins and CsgA were observed, a range of colony colors was noted, with the CsgAₛₛ-Pub1 cells staining as red as the CsgAₛₛ-NM cells and the CsgAₛₛ-Cyc8 cells staining only slightly darker than the negative control cells (data not shown).

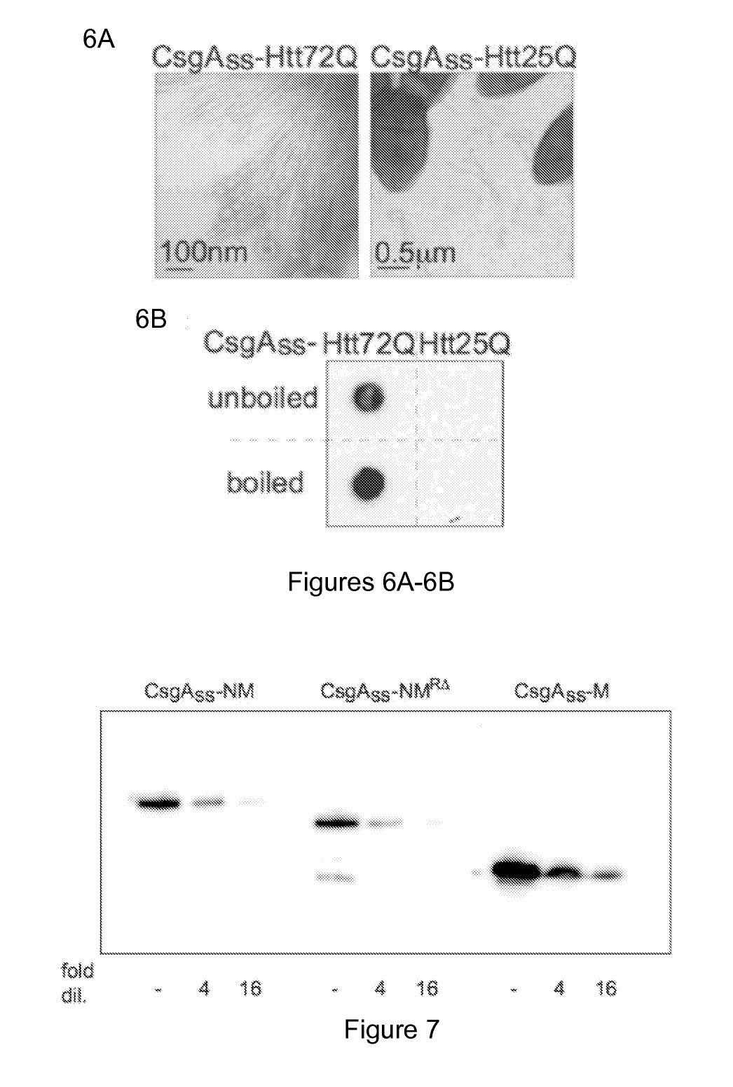

Having demonstrated that six different amyloidogenic yeast proteins form extracellular amyloid fibrils when directed to the curli export apparatus, a disease-associated mammalian amyloid-forming protein, exon 1 of the human huntingtin protein (Htt), was examined. The amyloidogenicity of Htt depends on the number of glutamines within the so-called polyQ region (Scherzinger et al. 1997). Thus, both a pathogenic polyQ-expansion variant of Htt exon 1 (Htt72Q (“72Q” disclosed as SEQ ID NO: 9)) and a nonpathogenic variant (Htt25Q (“25Q” disclosed as SEQ ID NO: 10)) were fused to the CsgAₛₛ and provided with a His₆-tag (SEQ ID NO: 11) at the C-terminus. Cells exporting CsgAₛₛ-Htt72Q (“72Q” disclosed as SEQ ID NO: 9) produced an abundance of fibrils organized into fan-like structures (FIG. 6A), whereas no fibrils were observed with Htt25Q (“25Q” disclosed as SEQ ID NO: 10). This fibrillar material bound CR and exhibited apple-green birefringence when examined between cross polarizers (data not shown). Finally, the aggregates were SDS-stable; strikingly, they remained SDS-stable even when the samples were boiled, a property that is characteristic of Htt amyloid aggregates (FIG. 6B) (Scherzinger et al. 1997).

Curli-Based Genetic Screen Enables Identification of an Amyloidogenic Protein from E. coli.

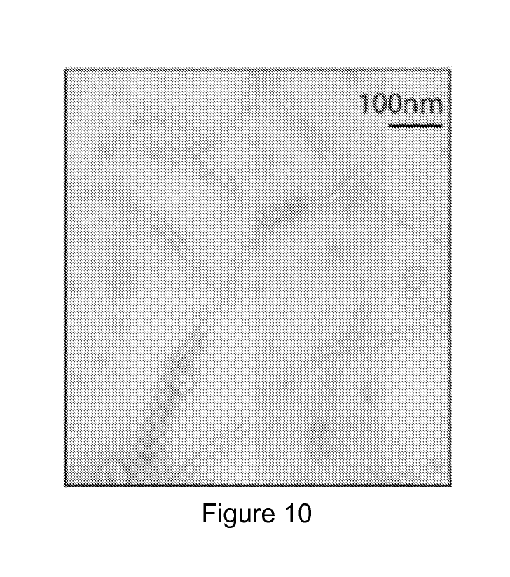

These findings indicate that amyloidogenic proteins (but not those that are non-amyloidogenic) readily form extracellular amyloid fibrils when secreted via the curli export pathway. In principle, therefore, the curli-based export system described herein should provide a convenient method for carrying out unbiased screens to identify amyloidogenic proteins from genomic or cDNA libraries. As a preliminary test of the possibility, a pilot screen was performed using a pool of approximately 614 E. coli ORFs (˜ 1/7 of the complete ORF library) (Saka et al. 2005). Universal primers that enabled the fusion of the collection of ORFs to the CsgAₛₛ and provision of a His₆-tag (SEQ ID NO: 11) at the C-terminus were used. The resulting library of plasmids directing the arabinose-inducible synthesis of these fusion proteins were used to transform ΔcsgBAC cells already containing the CsgG plasmid and the transformants plated on inducing medium supplemented with CR. ˜10,000 transformants were examined two particularly bright red colonies that resembled those formed by cells exporting CsgAₛₛ-NM were identified. DNA sequence analysis revealed that both of these transformants contained the same plasmid encoding fliE. A component of the flagellar basal body, FliE is not known to form amyloid under physiological conditions; however, previous work indicates that FliE readily forms amyloid fibrils in vitro (Saijo-Hamano et al. 2004). Consistently, cells exporting the CsgAₛₛ-FliE fusion protein revealed an abundance of fibrillar aggregates when examined by transmission EM (FIG. 10). The results described herein demonstrate that the curli-based export system can be exploited as a means to screen genomic libraries for amyloidogenic proteins.

Discussion

The results described herein indicate that heterologous amyloid-forming proteins from yeast, humans and bacteria readily adopt the amyloid fold when secreted via the E. coli curli export apparatus. 6 yeast proteins were tested, all of which are capable of assembling as amyloid fibrils in vitro (Alberti et al. 2009) and each formed extracellular amyloid fibrils upon export via the system described herein. Furthermore, in the case of the well-characterized yeast prion protein Sup35, it was demonstrated that the protein accesses an infectious prion conformation. In addition, the human Htt protein was tested and a pathogenic polyQ expansion variant (Htt72Q (“72Q” disclosed as SEQ ID NO: 9)) formed amyloid fibrils when exported by the E. coli cells, whereas a nonpathogenic variant (Htt25Q (“25Q” disclosed as SEQ ID NO: 10)) did not. Finally, a pilot screen performed with a partial E. coli ORF library establishes the feasibility of using this curli-based export system as a platform to screen for amyloidogenic proteins from genomic or cDNA libraries.

Secretion Via the Curli Export Pathway Facilitates Acquisition of the Amyloid Fold.

The findings described herein indicate that passage of an amyloidogenic protein through the CsgG pore facilitates its assembly into amloid fibrils in the extracellular milieu. Without wishing to be bound by theory, the passage of substrate proteins through the export pore in a relatively unfolded conformation (Robinson et al. 2006; Nerminger et al. 2011) and their accumulation to a relatively high local concentration in the extracellular milieu could facilitate their amyloid aggregation. Additionally, the lipid environment at the cell surface may be a contributing factor; lipids have been shown previously to facilitate conversion of recombinant PrP to an infectious amyloid conformation (Wang et al. 2010).

The results described herein further suggest that the curli export process facilitates amyloid conversion for proteins that ordinarily access the amyloid conformation only under restrictive conditions and/or rarely. In particular, the spontaneous conversion of Sup35 NM to the prion form in yeast cells is strictly dependent on the presence of a PIN factor and occurs only rarely (Liebman and Chernoff 2012; Derkatch et al. 1997; Derkatch et al. 2001; Osherovich and Weissman 2001). In contrast, secretion through the curli export apparatus circumvents the requirement for a PIN factor.

A Cell-Based Method for Evaluating Amyloidogenicity.

The findings described herein suggest that the curli export system can serve as a general cell-based method for producing amyloid aggregates and distinguishing amyloidogenic proteins from those that do not readily undergo conversion to an amyloid state. This system, termed a CDAG (curli-dependent amyloid generator), provides a convenient alternative to widely used in vitro assays for studying amyloid aggregation. In particular, CDAG provides an efficient method for evaluating amyloid-forming potential without a need for protein purification.

In principle, CDAG should facilitate the implementation of high throughput screens for identifying amyloidogenic proteins and modulators of amyloid aggregation. The results of the pilot screen performed using a partial E. coli ORF library imply that plating the cells on solid medium containing CR can identify amyloidogenic proteins that bind CR efficiently. Furthermore, the findings with both strong and weak CR binders suggest that the use of the filter retention assay as a primary screening step should reliably identify an even broader spectrum of amyloidogenic proteins.

Several genome-wide screens have been carried out in yeast in order to identify new prion proteins and prion protein candidates. An essential Q/N-rich region found in the originally identified yeast prion proteins was exploited in developing algorithms to identify additional prion proteins (Sondheimer and Lindquist 2000; Santoso et al. 2000; Michelitsch and Weissman 2000). More recently, using a variant bioinformatic approach, Alberti et al. (2009) identified some 200 proteins in S. cerevisiae with candidate Q/N-rich prion-forming domains; among the 100 that were examined experimentally approximately ¼ were found to contain a bona fide prion-forming domain. Taking a strictly genetic approach, Suzuki et al. (2012) performed a functional genome-wide screen by identifying yeast ORFs that could serve as PIN factors for a synthetic Sup35 variant; this screen uncovered a new prion that lacks the Q/N-rich signature region. As an unbiased screening platform, CDAG can permit the discovery of novel classes of prion-like or other amyloidogenic proteins because the export process can enable bypass of restrictive conditions for amyloid conversion.

In addition to identifying amyloidogenic proteins, CDAG can facilitate the identification of modulators of amyloid aggregation. Thus, for any particular amyloid-forming protein that binds CR when assembled into amyloid fibrils, the use of CR-containing medium would facilitate the identification of mutations or small molecules that hinder or accelerate the conversion process and the filter retention assay would provide a secondary screening step.

A Potential Means to Interrogate the Amylome.

Over the past decade diverse computational approaches have been developed for predicting amyloid-forming propensity in order to define the amylome--the proteome subset capable of forming amyloid-like fibrils (Goldschmidt et al. 2010). These include both sequence-based approaches (Fernandez-Escamilla et al. 2004; Trovato et al. 2006; Tartaglia et al. 2008; Bryan et al. 2009; Maurer-Stroh et al. 2010; O'Donnell et al. 2011) and structure-based approaches (Thompson et al. 2006; Zhang et al. 2007; Goldschmidt et al 2010), the latter of which are designed to identify short amyloidogenic motifs based on their steric zipper-forming potential (Sawaya et al. 2007). In particular, the Eisenberg group has used 3D profiling to evaluate short (6-residue) protein segments to identify high fibrillation propensity (HP) segments. Importantly, recent work indicates that despite the prevalence of HP segments (most proteins contain at least one), their ability to induce protein fibrillation is highly context dependent (Goldschmidt et al 2010). Specifically, such segments must be surface exposed with sufficient conformational flexibility to drive fibrillation. Despite the progress that has been made in predicting amyloid-forming propensity, significant challenges remain, especially when no structural information is available. Additionally, different algorithms appear to be differentially suited for the identification of different classes of amyloid-forming proteins (Toombs et al. 2012). Furthermore, experimental methods that can be used for algorithm validation by rapidly assessing amyloidogenicity on a genome-wide scale are lacking. The CDAG system described herein can detect amyloid fibril formation under a uniform and physiologically relevant set of conditions, and is thus particularly well suited for generating comprehensive datasets against which to test and refine computational models, thereby extending the understanding of the nature of amyloid formation.

Materials and Methods

Strains, Plasmids and Cell Growth.

A complete list of strains and plasmids is provided in Table 2. E. coli strain VS16 was constructed by replacing the csgBAC genes of strain MC4100 with a kanamycin-resistance gene using a previously described protocol (Datsenko and Wanner 2000). CsgG was produced under the control of the lacUV5 promoter on plasmid pVS76. Export-directed fusion proteins contain the first 42 residues of CsgA at the N-terminus and a His₆ tag (SEQ ID NO: 11) at the C-terminus and are produced under the control of the arabinose inducible P_{BAD} promoter. For fibril production, overnight cultures of VS16 transformed with compatible plasmids directing the synthesis of CsgG and an export-directed fusion protein were diluted to OD₆₀₀ 0.01 in LB supplemented with the appropriate antibiotics (Carbenicillin 100 μg/ml; Chloramphenicol 25 μg/ml). After 30 minutes of growth at 37° C., 5 μl of the culture was spotted on LB agar plates supplemented with the appropriate inducers (L-Arabinose at 0.2% w/v; IPTG at 1 mM), antibiotics (Carbenicillin 100 μg/ml; Chloramphenicol 25 μg/ml) and, where indicated, CR (5 μg/ml). Plates were then incubated at room temperature for 120 hr.

Scraped Cell Suspension Preparation.

To prepare unlysed cell suspensions, cells that had been spotted on agar were scraped off of the plates in PBS (phosphate-buffered saline) and normalized to OD₆₀₀ 1.0 in a volume of 100 μl. To prepare lysed cell suspensions, BUGBUSTER® PROTEIN EXTRACTION REAGENT™ (Novagen), rlysozyme (Novagen) and OMNICLEAVE™ endonuclease (Epicentre) were added to the unlysed cell suspensions to final concentrations of 0.5×, 300 units/ml and 10 units/ml, respectively, followed by incubation at room temperature with gentle rocking for 15 min. SDS was then added to 2% v/w. Boiled samples were incubated at 98° C. for 20 min after the addition of SDS.

Filter Retention Assay.

The filter retention assay was performed as previously described (Garrity et al. 2010). Lysed cell suspensions were filtered through the membrane in a volume of 200 μl. The membrane was probed with either anti-Sup35 (yS-20; Santa Cruz Biotechnology) or anti-His₆ (“His₆” disclosed as SEQ ID NO: 11) (clone His-2; Roche) to detect immobilized protein.

Extract Seeding Assay.

The extract seeding assay was performed as previously described (Garrity et al. 2010) with the exception that samples used as seeds were unlysed cell suspensions (if bacterial) or cell extracts (if of yeast origin). Yeast extracts were prepared as previously described (Garrity et al. 2010).

Yeast Transformations.

Protein transformations were performed essentially as previously described (Garrity et al. 2010) with the following modifications. To test the infectivity of unlysed cell suspensions, tetracycline (10 μg/ml) was added to the selection plate to inhibit bacterial growth. To prepare transformation samples consisting of polymerized material from the seeding assay, aliquots of the various seeded reactions from the 30 min time point were centrifuged at 10000×g for 15 mins at 4° C., washed in 500 μl STC (1 M Sorbitol, 10 mM Tris pH 7.5, 10 mM CaCl₂), centrifuged again at 10000×g for 15 min at 4° C. and resuspended in 500 μl STC. Each resuspension was then split into two samples. One sample was subjected to sonication (Sonics Vibracell Microtip sonicator, 25% amplitude, pulsed 1 s “on” and 3 s “off” for a total of 10 s of “on” time), and both samples were then used to transform [pin⁻ ][psi⁻] psi yeast cells.

Electron Microscopy and Immunolabeling.

Unlysed cell suspensions were adsorbed onto carbon or formvar/carbon-coated nickel grids in PBS, washed by floating the grid on 10 μl of distilled water, blotted dry, negatively stained with 1% uranyl acetate, blotted dry, and then viewed on a JEOL 1200EX™ microscope at an accelerating voltage of 80 KV. To immunolabel fibers, sample-adsorbed nickel grids were floated on blocking buffer consisting of 1% BSA (bovine serum albumin) in PBS for 15 min. Samples containing CsgAₛₛ-NM and CsgAₛₛ-M were then incubated with anti-Sup35 antibody (diluted 1:20), whereas samples containing CsgAₛₛ-Htt72Q (“72Q” disclosed as SEQ ID NO: 9) or CsgAₛₛ-Htt25Q (“25Q” disclosed as SEQ ID NO: 10) were incubated with anti-His₆ (“His₆” disclosed as SEQ ID NO: 11) antibody (diluted 1:100) for 2 hrs in blocking buffer and rinsed in PBS. Samples exposed to anti-Sup35 were incubated with donkey anti-goat 12 nm gold secondary antibody (Jackson Immunoresearch Labs) for 1 hr before rinsing in PBS, whereas samples exposed to anti-His₆ (“His₆” disclosed as SEQ ID NO: 11) were incubated with Protein-A gold 10 nm (CMC-UMC, Utrecht) for 1 hr before rinsing in PBS. All grids were stained with 1% uranyl acetate. Images were taken with an AMT 2 k CCD camera.

Congo Red Birefringence.

Unlysed cell suspensions prepared using cells grown on CR-containing agar were spotted on a glass slide. Poly-L-lysine-coated cover slips were then placed on the samples and samples viewed between cross polarizers on a Nikon 80i upright microscope with a Plan Apo 100×1.4NA objective. Images were acquired using a Nikon Digital Sight DS-Fil™ color camera and NIS-ELEMENTS™ acquisition software.

Library Construction and Screen.

An expression library of csgAₛₛ fusions (with a C-terminal His₆ tag (SEQ ID NO: 11)) representing all the open reading frames (ORFs) from the E. coli ORF library (Saka at al. 2005) was constructed using a previously described method (Gibson et al. 2009). The library was constructed in a pooled format, with each pool representing approximately a seventh of the entire expression library. One pool from plasmid library was then transformed into strain VS16 that already contained the csgG overexpression plasmid, pVS76, and plated on LB agar supplemented with inducers (L-Arabinose at 0.2% w/v; IPTG at 1 mM), antibiotics (Carbenicillin 100 μg/ml; Chloramphenicol 25 μg/ml) and CR (5 μg/ml). Plates were then incubated at room temperature for 120 hr.

### REFERENCES

Alberti S, Halfmann R, King O, Kapila A, Lindquist S. 2009. A systematic survey identifies prions and illuminates sequence features of prionogenic proteins. Cell 137:146-158.
Bach S, Talarek N, Andrieu T, Vierfond J, Mettey Y, Galons H, Dormont D, Meijer L, Cullin C, Blondel M. 2003. Isolation of drugs active against mammalian prions using a yeast-based screening assay. Nat Biotech 21:1075-81.
Bagriantsev S N, Kushnirov V V, Liebman S W. 2006. Analysis of amyloid aggregates using agarose gel electrophoresis. Methods Enzymol 412:33-48.
Blanco L P, Evans M L, Smith D R, Badtke M P, Chapman M R. 2011. Diversity, biogenesis and function of microbial amyloids. Trends Microbiol 20:66-73.
Bryan A W, Menke M, Cowen L J, Lindquist S L, Berger B. 2009. BETASCAN: Probably β-amyloids identified by pairwise probabilistic analysis. PLoS Comput Biol 5:e1000333.
Chapman M R, Robinson L S, Pinkner J S, Roth, R, Heuser J, Hammar M, Normark S, Hultgren S J. 2002 Role of Escherichia coli curli operons in directing amyloid fiber formation. Science 295:851-55.
Chiti F, Dobson C M. 2006. Protein misfolding, functional amyloid, and human disease. Annu Rev Biochem 75:333-366.
Datsenko K A, Wanner B L. 2000. One-step inactivation of chromosomal genes in Escherichia coli K-12 using PCR products. Proc Natl Acad Sci USA 97:6640-45.
Derkatch I L, Bradley M E, Hong J Y, Liebman S W. 2001. Prions affect the appearance of other prions: the story of [PIN+]. Cell 106:171-182.
Derkatch I L, Bradley M E, Zhou P, Chernoff Y O, Liebman S W. 1997. Genetic and environmental factors affecting the de novo appearance of the [PSI⁺] prion in Saccharomyces cerevisiae. Genetics 147:507-519.
Fernandez-Escamilla A-M, Rousseau F, Schymkowitz J, Serrano L. 2004. Prediction of sequence-dependent and mutational effects on the aggregation of peptides and proteins. Nat Biotech 22:1302-1306.
Garrity S J, Sivanathan V, Dong J, Lindquist S, Hochschild A. 2010. Conversion of a yeast prion protein to an infectious form in bacteria. Proc Natl Acad Sci USA 107:10596-10601.
Gibson D G, Young L, Chuang R, Venter C J, Hutchison C A, Hamilton S O. 2009. Enzymatic assembly of DNA molecules up to several hundred kilobases. Nat Methods 6:343-45.
Glover J R, Kowal A S, Schirmer E C, Patino M M, Liu J J, Lindquist S. 1997. Self-seeded fibers formed by Sup35, the protein determinant of [PSI⁺], a heritable prion-like factor of S. cerevisiae. Cell 89:811-819.
Goldschmidt L, Teng P K, Riek R, Eisenberg D. 2010. Identifying the amylome, proteins capable of forming amyloid-like fibrils. Proc Natl Acad Sci USA 107:3487-92.
Hammar M, Arnqvist A, Bian Z, Olsen A, Normark S. 1995. Expression of two csg operons is required for production of fribronectin- and Congo red-binding curli polymers in Escherichia coli K-12. Mol Microbiol 18:661-670.
Hammer N D, Schmidt J C, Chapman M R 0.2007. The curli nucleator protein, CsgB, contains an amyloidogenic domain that directs CsgA polymerization. Proc Natl Acad Sci USA 104:12494-499.
Kim W, Kim Y, Kim D J, Chang Y-T, Hecht M H. 2006. A high-throughput screen for compounds that inhibit aggregation of the Alzherimer's peptide. ACS Chem Biol 7:461-69.
King C, Diaz-Avalos R. 2004. Protein-only transmission of three yeast prion strains. Nature 428:319-23.
Lee L L, Ha H H, Chang Y-T, DeLisa M P. 2009. Discovery of amyloid-beta aggregation inhibitors using an engineered assay for intracellular protein folding and solubility. Prot Sci 18:277-86.
Li L, Lindquist S. 2000. Creating a protein-based element of inheritance. Science 287:661-664.
Liebman S W, Chernoff Y O. 2012. Prions in yeast. Genetics 191:1041-72.
Liu J J, Lindquist S. 1999. Oligopeptide-repeat expansions modulate ‘protein-only’ inheritance in yeast. Nature 400:573-576.
Maji S K, Perrin M H, Sawaya M R, Jessberger S, Vadodaria K, Rissman R A, Singru P S, Nilsson K P R, Simon R, Schubert D, et al. 2009. Functional amyloids as natural storage of peptide hormones in pituitary secretory granules. Science 325:328-332.
Maurer-Stroh S, Debulpaep M, Kuemmerer N, Lopez de la Paz M, Martins I C, Reumers J, Morris K L, Copland A, Serpell L, Serrano L et al. 2010. Exploring the sequence determinants of amyloid structure using position-specific scoring matrices. Nat Meth 7: 237-42.
Michelitsch M D, Weissman J S. 2000. A census of glutamine/asparagine-rich regions: implications for their conserved function and prediction of novel prions. Proc Natl Acad Sci USA 97:11910-915.
Henninger A A, Robinson L S, Hammer N D, Epstein E A, Badtke M P, Hultgren S J, Chapman M R. 2011. CsgE is a curli secretion specificity factor that prevents amyloid fibre aggregation. Mol Microbiol 81: 486-99.
O'Donnell C W, Waldispuhl J, Lis M, Halfmann R, Devadas S, Lindquist S, Berger B. 2011. A method for probing the mutational landscape of amyloid structure. Bioinformatics 27: i34-i42.
Osherovich L Z, Weissman J S. 2001. Multiple Gln/Asn-rich prion domains confer susceptibility to induction of the yeast [PSI+] prion. Cell 106:183-194.
Robinson L S, Ashman E M, Hultgren S J, Chapman M R. 2006. Secretion of curli fibre subunits is mediated by the outer membrane-localized CsgG protein. Mol Microbiol 59:870-881.
Saijo-Hamano Y, Uchida N, Namba K, Oosawa K. 2004. In vitro characterization of FlgB, FlgC, FlgF, FlgG, and FlgE, flagellar basal body proteins of Salmonella. J Mol Biol 339:423-35.
Saka K, Tadenuma M, Nakade S, Tanaka N, Sugawara H, Nishikawa K, Ichiyoshi N, Kitagawa M, Mori H, Ogasawara N, Nishimura A. 2005. A complete set of Escherichia coli open reading frames in mobile plasmids facilitating genetic studies. DNA Res 12:63-68.
Santoso A, Chien P, Osherovich L Z, Weissman J S. 2000. Molecular basis of a yeast prion species barrier. Cell 100:277-88.
Sawaya M R, Sambashivan S, Nelson R, Ivanova M I, Sievers S A, Apostol M I, Thompson M J, Balbirnie M, Wiltzius J J W, McFarlane H T et al. 2007. Atomic structures of amyloid cross-β spines reveal varied steric zippers. Nature 447:453-57.
Scherzinger E, Lurz R, Turmaine M, Mangiarini L, Hollenbach B, Hasenbank R, Bates G P, Davies S W, Lehrach H, Wanker E E. 1997. Huntingtin-encoded polyglutamine expansions form amyloid-like protein aggregates in vitro and in vivo. Cell 90:549-558.
Sondheimer N, Lindquist S. 2000. Rnq1: an epigenetic modifier of protein function in yeast. Mol Cell 5:163-172.
Suzuki G, Shimazu N, Tanaka M. 2012. A yeast prion, Mod5, promotes acquired drug resistance and cell survival under environmental stress. Science 336: 355-59.
Tanaka M, Weissman J S. 2006. An efficient protein transformation protocol for introducing prions into yeast. Methods Enzymol 412:185-200.
Tartaglia G G, Pawar A P, Campioni S, Dobson C M, Chiti F, Vendruscolo M. 2008. Prediction of aggregation-prone regions in structured proteins. J Mol Biol 380:425-436.
Teng P K, Eisenberg D. 2009. Short protein segments can drive a non-fibrillizing protein into the amyloid state. Protein Eng Des Sel 22:531-536.
Thompson M J, Sievers S A, Karanicolas J, Ivanova M I, Baker D, Eisenberg D. 2006. The 3D profile methods for identifying fibril-forming segments of proteins. Proc Natl Acad Sci USA 103:4074-4078.
Toombs J A, Petri M, Paul K R, Kan G, Ben-Hur A, Ross, Eric D. (2012) De novo design of synthetic prion domains. Proc Natl Acad Sci USA 109:6519-24.
Toyama B H, Weissman J S. 2011. Amyloid structure: conformational diversity and consequences. Annu Rev Biochem 80:557-585.
Trovato A, Chiti F, Maritan A, Seno F. 2006. Insight into the structure of amyloid fibrils from the analysis of globular proteins. PLoS Comput Biol 2:e170.
Tuite M F and Serio T R. 2010. The prion hypothesis: from biological anomaly to basic regulatory mechanism. Nat Rev Mol Cell Biol 12:823-833
Wang F, Wang X, Yuan C-G, Ma J. 2010. Generating a prion with acterially expressed recombinant prion protein. Science 327: 1132-35.
Wang X, Chapman M R. 2008. Sequence determinants of bacterial amyloid formation. J Mol Biol 380:570-80.
Wang X, Smith D R, Jones J W, Chapman M R. 2007. In vitro polymerization of a functional Escherichia coli amyloid protein. J Biol Chem 282:3713-3719.
Wickner R B, Edskes H K, Shewmaker F, Nakayashiki T. 2007. Prions of fungi: inherited structures and biological roles. Nat Rev Microbiol 5:611-618.
Zhang Z, Chen H, Lai L 0.2007. Identification of amyloid fibril-forming segments based on structure and residue-based statistical potential. Bioinformatics 23:2218-2225.

TABLE 1
Infection of [pin−−][psi−−] yeast spheroplast with scraped cell
samples (CsgAss--NM cells or CsgAss--M). Analysis of the data
using Fisher's exact test suggests that the observed difference in the
frequencies of [PSI+] transformants is statistically significant (P = 10⁻³).
Transformation of [psi⁻][pin⁻] yeast with scraped cell suspensions

| | |
|---|---|
| Phenotype of yeast | exported fusion protein: |

| after transformation | CsgAₛₛ-NM | CsgAₛₛ-M |
|---|---|---|
| [PSI⁺] | 9 | 0 |
| [psi⁻] | 2192 | 1844 |

TABLE 2
(“His₆” disclosed as SEQ ID NO: 11, “25Q” disclosed as SEQ
ID NO: 10 and “72Q” disclosed as SEQ ID NO: 9)

| Strain/plasmid | Genotype or relevant characteristics | Source/Ref |
|---|---|---|
| Strain | | |
| Escherichia coli | | |
| MC4100 | F-, [araD139]_{B/r}, Δ(argF-lac)169, λ⁻, e14-, flhD5301, | Casadaban |
| | Δ(fruK-yeiR)725(fruA25), relA1, rpsL150(strR), | 1976 |
| | rbsR22, Δ(fimB-fimE)632(::lS1), deoC1 | |
| VS16 | MC4100 Δ(csgBAC)(::kanR) | This study |
| Saccharomyces cerevisiae | | |
| SG775 | YJW187 [pin⁻]; derived by serial passage on YPD | Garrity et al. |
| | with 3 mM GuHCl; phenotypically [pin⁻][psi⁻] | 2010 |
| Plasmid | | |
| pVS59 | bla P_{BAD} csgA-His₆, pBR322 ori; produces CsgA | This study |
| | fused to His₆. | |
| pVS76 | cat Pl_{acDVS} csgG, pACYC184 ori, produces CsgG | This study |
| All plasmids | bla P_{BAD} csgAₛₛ-[test protein]-His₆, pBR322 ori; | This study |
| listed below | produces CsgA residues 1-42 fused to a test | |
| | protein with a C-terminal His₆-tag. See below for | |
| | the test proteins used in this study | |
| pVS72 | Sup35NM (residues 1-253) | This study |
| pVS87 | New1 (residues 50-100) | This study |
| pVS88 | Sup35NM^{R}(residues 1-253 with oligopeptide | This study |
| | repeats 2-5 deleted) | |
| pVS105 | Sup35M (residues 125-253) | This study |
| pVS116 | Cyc8 (residues 442-678) | This study |
| pVS117 | Mss11 (residues 270-429) | This study |
| pVS118 | Pub1 (residues 243-327) | This study |
| pVS119 | Rnq1 (residues 153-405) | This study |
| pVS189 | Htt25Q (Huntingtin exon 1 with a 25-residue poly- | This study |
| | Q segment) | |
| pVS190 | Htt72Q (Huntingtin exon 1 with a 72-residue poly- | This study |
| | Q segment) | |
| pVS230 | Snf2 (residues 45-240) | This study |
| pVS231 | Med2 (residues 280-366) | This study |

Casadaban M J. 1976. Transposition and fusion of the lac genes to selected promoters in Escherichia coli using bacteriophage lambda and Mu. J Mol Biol 104: 541-555
Garrity S J, Sivanathan V, Dong J, Lindquist S, Hochschild A. 2010. Conversion of a yeast prion protein to an infectious form in bacteria. Proc Natl Acad Sci USA 107: 10596-10601.

1. A prokaryotic cell comprising:
a nucleic acid sequence encoding a recombinant polypeptide, the recombinant polypeptide comprising, from 5′ to 3′ a bipartite curli signal sequence and a heterologous polypeptide sequence;
wherein the bipartite curli signal sequence comprises, from 5′ to 3′ a SecA-dependent secretion signal and a CsgG targeting sequence.

2. The cell of claim 1, wherein the SecA-dependent secretion signal comprises the polypeptide sequence of SEQ ID NO: 1 (CsgA) or SEQ ID NO: 2 (CsgB).

3. The cell of claim 1, wherein the CsgG targeting sequence comprises the polypeptide sequence of SEQ ID NO: 3 or SEQ ID NO: 4.

4. The cell of claim 1, further comprising,
a nucleic acid encoding a CsgG polypeptide wherein the CsgG polypeptide is expressed at ectopic expression levels.

5. The cell of claim 1, wherein the cell has been engineered to not transcribe or translate a csgA or csgB gene.

6. (canceled)

7. The cell of claim 1, wherein the heterologous polypeptide sequence is selected from the group consisting of:
PrP; AP; α-synuclein; Sup35; the NM domain of Sup35; Rnq1; Cyc8; New1; Mss11;
Pub1; Htt; exon 1 of Htt; NMRΔ; NMR2E2, FliE, Het-s; Tau; Superoxide dismutase 1;
Htt with polyQ expansion; ataxins with polyQ expansion; serum amyloid A;
transthyretin; fibrinogen; fibrinogen α-chain; amylin (IAPP); and amyloid aggregate-forming domains or fragments thereof.

8. The cell of claim 1, wherein an anchor sequence comprised by the heterologous polypeptide sequence has been replaced with the CsgB anchor sequence.

9. The cell of claim 1, wherein the nucleic acid sequence encoding a recombinant polypeptide further comprises a protease cleavage site sequence located between the bipartite curli signal sequence and the heterologous polypeptide sequence.

10. The cell of claim 1, wherein the nucleic acid sequence encoding a recombinant polypeptide further comprises, from 5′ to 3′ an amyloidogenic peptide sequence and a protease cleavage site sequence located between the bipartite curli signal sequence and the heterologous polypeptide sequence.

11. The cell of claim 10, wherein the amyloidogenic peptide sequence encodes Sup35NM.

12. (canceled)

13. (canceled)

14. (canceled)

15. A method of producing amyloidogenic polypeptides, comprising culturing the cell of claim 1 under conditions suitable for the expression and export of the recombinant polypeptide.

16. The method of claim 15, wherein an extracellular amyloid polypeptide aggregate comprises the amyloidogenic polypeptides.

17. (canceled)

18. (canceled)

19. (canceled)

20. (canceled)

21. The method of claim 15, wherein the cell is cultured in medium comprising an amyloid facilitating factor.

22. The method of claim 21, wherein the amyloid facilitating factor is selected from the group consisting of:
RNA; polyanions; POPG the synthetic anionic phospholipid; lipids; and amyloidogenic polypeptide seed material.

23. A method of determining if a candidate polypeptide sequence comprises an amyloidogenic polypeptide, the method comprising;
culturing the cell of claim 1 under conditions that permit the expression and export of the recombinant polypeptide;
determining the presence or absence of amyloid aggregates;
wherein the heterologous polypeptide sequence comprises the candidate polypeptide sequence;
wherein the presence of amyloid aggregates indicates the candidate polypeptide sequence comprises an amyloidogenic polypeptide.

24. The method of claim 23, wherein the cell is further cultured under conditions that permit the formation of amyloid aggregates.

25. The method of claim 24, wherein the conditions that permit the formation of amyloid aggregates comprise culturing the cell on solid medium.

26. The method of claim 23, wherein the cell is contacted with an amyloid-binding dye.

27. (canceled)

28. (canceled)

29. A method of identifying an amyloidogenic modulating agent, the method comprising;
culturing a cell of claim 1 under conditions that permit the expression and export of the recombinant polypeptide;
contacting the cell with a candidate agent;
determining if the formation of amyloid aggregates is modulated;
wherein a statistically significant difference in amyloid aggregation as compared to a reference indicates that the candidate agent is an amyloidogenic modulating agent.

30. (canceled)

31. The method of claim 29, wherein the cell is cultured in a liquid medium; whereby a) expression and export of the recombinant polypeptide is permitted and b) the formation of amyloid aggregates is inhibited.

32. The method of claim 29, wherein the heterologous polypeptide comprises a variant of an amyloidogenic polypeptide that forms amyloid aggregates at a lower rate than the wild-type amyloidogenic polypeptide.

33-60. (canceled)
